(19) United States
(12) Patent Application Publication
(10) Pub. No.: US 2019/0249147 A1
(43) Pub. Date: Aug. 15, 2019

(54) BIOLOGICALLY RELEVANT IN VITRO SCREENING OF HUMAN NEURONS

(71) Applicant: The Board of Trustees of the Leland Stanford Junior University, Stanford, CA (US)

(72) Inventors: Jonathan Davila, Sunnyvale, CA (US); Daniel Haag, Stanford, CA (US); Marius Wernig, Stanford, CA (US); Siddhartha S. Mitra, Aurora, CO (US); Thomas C. Sudhof, Stanford, CA (US)

(21) Appl. No.: 16/310,632

(22) PCT Filed: Jun. 20, 2017
(86) PCT No.: PCT/US17/38273

§ 371 Date: Dec. 17, 2018

Related U.S. Application Data
(60) Provisional application No. 62/352,343, filed on Jun. 20, 2016.

(51) Int. Cl. C12N 5/071; G01N 33/50

(52) U.S. Cl. CPC C12N 5/0697; G01N 33/5058; C12N 2501/60; C12N 2501/11; C12N 2502/086; C12N 2501/33; C12N 2502/081; C12N 2503/04; C12N 2501/155

(57) ABSTRACT

Compositions and methods are provided for biologically relevant in vitro screening of neural function, including determination of the effects of an agent on neural cells. The compositions of the invention useful in such screening methods include a neural co-culture system comprising human pluripotent stem cell (PSC)-derived neurons and human glial cells, which may be derived by culture methods allowing for rapid and robust development of highly mature neuronal activity, particularly spontaneous synchronous network bursts.

### CROSS REFERENCE

This application claims benefit and is a 371 application of PCT Application No. PCT/US2017/038273, filed Jun. 20, 2017, which claims benefit of U.S. Provisional Patent Application No. 62/352,343, filed Jun. 20, 2016, which applications are incorporated herein by reference in their entirety.

### BACKGROUND OF THE INVENTION

Pharmaceutical drug discovery utilizes the identification and validation of therapeutic targets, as well as the identification and optimization of lead compounds. The explosion in numbers of potential new targets and chemical entities resulting from genomics and combinatorial chemistry approaches over the past few years has placed massive pressure on screening programs. The rewards for identification of a useful drug are enormous, but the percentages of hits from any screening program are generally very low. Desirable compound screening methods solve this problem by both allowing for a high throughput so that many individual compounds can be tested; and by providing biologically relevant information so that there is a good correlation between the information generated by the screening assay and the pharmaceutical effectiveness of the compound.

Some of the more important features for pharmaceutical effectiveness are specificity for the targeted cell or disease, a lack of toxicity at relevant dosages, and specific activity of the compound against its molecular target. Therefore, one would like to have a method for screening compounds or libraries of compounds that allows simultaneous evaluation for the effect of a compound on the biologically relevant cell population, where the assay predicts clinical effectiveness.

The effect of drugs on neurons is of particular interest, where efficacy and toxicity may rest in sophisticated analysis of firing behavior, or the ability of neurons to form functional networks, rather than on simple viability assays. The discrepancy between the number of lead compounds in clinical development and approved drugs may partially be a result of the methods used to generate the leads and highlights the need for new technology to obtain more detailed and physiologically relevant information on cellular processes in normal and diseased states.

A number of important clinical conditions are associated with neuronal physiology, for example diseases such as Alzheimer's disease (AD), fragile X syndrome (FXS), Parkinson's disease (PD), Huntington's disease (HD), spinal muscular atrophy (SMA), multiple sclerosis (MS), and amyotrophic lateral sclerosis (ALS). Other conditions are manifest in cognitive function, and are likely to have an association with neuronal function and interaction, e.g. psychiatric conditions such as schizophrenia, bipolar disorders, attention deficit hyperactivity disorder (ADHD), depression; with developmental disorders including autism spectrum disorders, etc.

In addition to pharmaceutical drug discovery, there is a pressing need for meaningful screening platforms to identify and explore specific toxicity effects due to the increasing number of new therapeutic compounds and chemical substances with human exposure. Particularly, in the field of neurotoxicity, assays capable of assessing the impairment of neuronal function are still lacking for human cells.

Therefore, the development of in vitro screening platforms that recapitulate highly functional human tissue is of utmost importance. In order to study functional consequences of molecular interactions between compounds and targets as well as associated cellular mechanisms phenotypic readouts are indispensable. Consequentially, suitable in vitro screening platforms require the integration of highly specified cell types into a physiologically relevant functional system and the measurement of defined parameters.

### RELEVANT LITERATURE

U.S. Pat. No. 9,057,053 discloses methods for the differentiation of neurons from induced pluripotent cells. Chanda et al. (2014) Stem Cell Reports 3(2):282-96 discusses the generation of induced neuronal cells by the single reprogramming factor ASCL1. Zhang et al. (2013) Neuron 78(5):785-98 provides characterization of induced neurons generated from human pluripotent stem cells.

Geissler (2012) J Neurosci Methods. 204(2):262-72. A new indirect co-culture set up of mouse hippocampal neurons and cortical astrocytes on microelectrode arrays. Wainger et al. (2014) Cell Rep. 7(1):1-11, Intrinsic membrane hyperexcitability of amyotrophic lateral sclerosis patient-derived motor neurons. Simeone (2013) Neurobiol Dis. 54:68-81, Loss of the Kv1.1 potassium channel promotes pathologic sharp waves and high frequency oscillations in in vitro hippocampal slices.

### SUMMARY OF THE INVENTION

Compositions and methods are provided for biologically relevant in vitro screening of neural function, including determination of the effects of an agent on neural cells. The compositions of the invention useful in such screening methods include a neural co-culture system comprising human pluripotent stem cell (PSC)-derived neurons and human glial cells, which may be derived by culture methods allowing for rapid and robust development of highly mature neuronal activity, particularly spontaneous synchronous network bursts. The composition of the neural co-culture system features defined subtypes of neuronal cells generated through direct conversion of cell identity. The neural co-culture system may also feature human glial cells.

In some embodiments, a neural co-culture system is provided that further comprises one or more monitoring devices to measure parameters of neuronal activity. Monitoring device components may be designed for electrophysiology- and imaging-based detection methods, e.g. microelectrode arrays, amplifiers, cameras, data analysis systems, and the like. The combination of monitoring device and neural co-culture may be referred to herein as a neural screening system. Neuronal activity, e.g. synchronous firing, can be analyzed by extracellular electric currents and field potentials using microelectrode arrays (MEAs) or by changes of intracellular calcium (Ca) and voltage dependent probes using fluorescence microscopy imaging. The combination of the co-culture system with medium-to-high throughput technologies to measure changes in neuronal activity allows screening without invading the cells. A schematic of an exemplary system is shown in FIGS. 1A and 1B.

In some embodiments, a neural co-culture system is provided that comprises monitoring devices to measure parameters of metabolic activity, cell viability, neuronal health, cellular organelle composition, cellular organelle morphology, cellular organelle function, enzyme function, intra-cellular signaling, cell morphology, cellular trafficking, protein abundance, protein localization, protein conformation (monomer, oligomer, aggregate) in neurons and glial cells. Further parameters may include colorimetry, luminescence, or fluorescence-based signals from incorporated reporter systems, e.g. reporter constructs for gene activation, autophagic flux, ligand binding, protein dimerization, cellular organelle composition, enzyme function, and the like. Monitoring device components may be designed for imaging-based detection, fluorescence-based detection, luminescence-based detection, light absorption-based detection, and colorimetry-based detection, e.g. fluorescence microscopes, cameras, photometers, spectrometers, ELISA-readers, and the like.

In some embodiments, the in vitro neural co-culture system comprises defined mixtures of homogenous populations of human neurons generated through direct neuronal induction of pluripotent stem cells (induced neurons, iNs). Induced neurons can be one or more selected subtypes or defined mixtures of subtypes, where subtypes include, without limitation, GABAergic inhibitory neurons, glutamatergic excitatory neurons, cholinergic neurons, noradrenergic neurons, dopaminergic neurons, serotonergic neurons, sensory neurons, spinal motor neurons, peripheral neurons, cortical neurons, etc. The co-culture may further comprise human or animal-derived glial cells (such as mouse or rat-derived), which can be obtained, for example, by culture of primary tissue, generated through direct induction or stepwise differentiation of PSC, and the like. Glial cells may comprise astrocytes, oligodendrocytes, microglia and different developmental stages, as well as differentiation- and activation states. Critical to the function of the co-culture system is formation of functional neural networks capable of spontaneous synchronous firing that can be used for phenotypic screening and other purposes.

In some embodiments, the neuronal screening system of the invention is contacted with candidate agents and/or conditions, and assessed for alterations in parameters of interest, including without limitation synchronous network firing. In some embodiments, neural cells comprising genetic changes or variations are assessed for alterations in parameters of interest in the presence or absence of candidate agents. In some embodiments, neural cells comprising epigenetic changes or modulation of specific gene expression are assessed for alterations in parameters of interest in the presence or absence of candidate agents. Such parameters may include, without limitation, one or more of measurements indicative of general viability, cellular organelle function, morphology and composition, neuronal maturation, neuronal health, neuronal morphology, synaptic density, synaptic function, basic neuronal activity, synchronous firing of neuronal networks, specific patterns of neuronal activity, as well as abundance, conformation, and localization of specific proteins. In some embodiments, parameters of neuronal activity are measured in response or in the presence of electrical stimulation or optogenetic stimuli or perturbation of specific components of the neural co-culture or the complete neuronal network.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1B. Schematic overview of a human neural in vitro co-culture system comprising inhibitory neuronal cells, excitatory neuronal cells and astroglial cells mixed in a defined ratio. (FIG. 1A) shows 1 tissue culture dish coated with suitable substrate (typically matrigel, polyethylenimine and laminin, or polyornithine and laminin), in which there is 2 neuronal maintenance media (Neurobasal-A medium, B27 supplement, Glutamax [1 mM], NT3 [10 ng/ml], mouse laminin [200 ng/ml]+AraC [2 μM], 1% FBS). Cell types may include one or more of 3 GABAergic inhibitory type of induced neuron (iN) derived from human pluripotent stem cells; 4 glutamatergic excitatory type of induced neuron (iN) derived from human pluripotent stem cells; optionally 5 dopaminergic excitatory type of induced neuron (iN) derived from human pluripotent stem cells and 6 astroglial cell derived from human pluripotent stem cells through stepwise differentiation. (FIG. 1B) is a cross-section of a schematic setup for monitoring neuronal activity in neural co-cultures using multielectrode arrays (MEAs), constituting a neuronal screening system of the invention. (FIG. 1B) shows a tissue culture plate containing multielectrode wells 11 (commercially available e.g. from Axion BioSystems in formats of 12-wells, 24-wells, 48-wells, and 96-wells), with suitable substrate coating 12 depending on the surface material (typically polyethylenimine and laminin for plastic ware); and neuronal maintenance or recording medium 13 (e.g. artificial cerebrospinal fluid, ACSF: 124 mM NaCl, 2.5 mM KCl, 2.5 mM CaCl₂, 21 mM MgSO₄, 26 mM NaHCO₃, 0.45 mM NaH₂PO₄—H₂O, 0.5 mM Na2HPO₄, 10 mM glucose, 4 mM sucrose). Optionally the system comprises a grid of microelectrodes 14 incorporated in the bottom of the tissue culture well. A circuit path 15 conducts the electrical signals from the electrode to the amplifier. Cell types may include one or more of 3 GABAergic inhibitory type of induced neuron (iN) derived from human pluripotent stem cells; 4 glutamatergic excitatory type of induced neuron (iN) derived from human pluripotent stem cells; optionally 5 dopaminergic excitatory type of induced neuron (iN) derived from human pluripotent stem cells and 6 astroglial cell derived from human pluripotent stem cells through stepwise differentiation.

FIG. 2A(i)-2B. (FIG. 2A(i)) Schematic representation indicating different categories of groups of spikes including bursts, synchronous firing, and network bursts. (FIG. 2A(ii)) Raster plot representation depicting spike signals per electrode (y-axis) over time (x-axis) of synchronized network bursts from co-cultures on multielectrode array (MEA) plates. (FIG. 2B) Schematic diagram to illustrate common parameters of neuronal activity measured on MEAs (modified from Axion BioSystems).

FIG. 3A-3B. Detection of synchronized neuronal network activity in co-cultures of primary glial cells and glutamatergic excitatory iN cells measured on MEAs. (FIG. 3A) Raster plots showing the development of synchronized network bursts over time after plating excitatory iN cells together with glial cells. (FIG. 3B) Raster plots showing neuronal network activity in response to electrical stimulation.

FIG. 4A-4B(vi). Formation of spontaneous synchronized network activity in a co-culture of primary glial cells and glutamatergic excitatory iN cells. (FIG. 4A) Raster plots showing development of synchronous network bursts (indicated by pink boxes). (FIG. 4B(i)-4B(vi)) Quantification of basic parameters describing general activity (mean firing rate and number of active electrodes), bursting (frequency of bursts and duration of bursts), and synchrony (percentage of bursts occurring within network bursts and cross-correlation between spikes detected by different electrodes across each well).

FIG. 5A-5B(i)-5B(vi). Formation of spontaneous synchronized network activity in a co-culture of primary glial cells and a combination of inhibitory and excitatory iN cells. (FIG. 5A) Raster plots showing development of synchronous network bursts. (FIG. 5B(i)-5B(vi)) Quantification of basic parameters describing general activity, bursting, and synchrony.

FIG. 6A-6D(vi). Effects of chemical compounds on neuronal network activity in neural co-cultures consisting of primary glial cells and glutamatergic excitatory iN cells. (FIG. 6A-6A(vi)) Raster plots and quantification of baseline activity (before treatment) and dosed activity (after treatment) for the compound solvent DMSO (control experiment). (FIG. 6B-6B(vi)) Raster plots and quantification of baseline activity and dosed activity for the AMPA-receptor antagonist CNQX. (FIG. 6C-6C(vi)) Raster plots and quantification of baseline activity and dosed activity for the NMDA-receptor antagonist AP5. (FIG. 6D-6D(vi)) Raster plots and quantification of baseline activity and dosed activity for the GABA-receptor antagonist PTX.

FIG. 7A-7B(i). Effects of chemical compounds on neuronal network activity in neural co-cultures consisting of primary glial cells and a mixture of glutamatergic excitatory iN cells and GABAergic inhibitory iN cells. (FIG. 7A(i)-7A(vi)) Compound treatment of neural co-cultures reflecting an approximate ratio of 70%/30% for excitatory/inhibitory cells. Raster plots and quantification of baseline activity and dosed activity for the GABA-receptor antagonist PTX. (FIG. 7B-7B(i)) Compound treatment of neural co-cultures reflecting an approximate ratio of 50%/50% for excitatory/inhibitory cells. Raster plots of baseline activity and dosed activity for PTX.

FIG. 8A-8B. Synchronized network activity in neural co-cultures containing either primary glial cells derived from mice or human glial cells differentiated from early glial progenitors. (FIG. 8A) Raster plots showing different frequencies of synchronized network bursts (same time scale) and reduced firing between bursts in co-cultures using human versus mouse glial cells. (FIG. 8B) Patch clamp analysis measuring excitatory postsynaptic currents (EPSCs) of single neurons from co-cultures using human glial cells (left) or mouse glial cells (right).

FIG. 9A-9E. Effects of well-described neurotoxicants on overall neuronal spiking behavior and synchronized network activity. (FIG. 9A-9C) Raster plots and quantification of multiple neuronal activity parameters measured on multielectrode arrays after exposing neural co-cultures to different concentrations of well-established neurotoxic chemicals. Measurements are plotted as changes over baseline recording. (FIG. 9D-9E) Comparison of the effects of well-established neurotoxic compounds on neuronal activity and cell viability between primary rat cortical cultures and human iN neuronal co-cultures. Changes in spiking rates (mean firing rate, MFR) and viability show high concordance between both species and culture types.

FIG. 10A-10B. Effects of proconvulsive compounds on neuronal network activity and application of countermeasures. (FIG. 10A) Exposure of the human neural co-culture to the GABAA-receptor antagonist bicuculline induces ictal-like discharges that mimic neuronal firing during seizures and changes specific network activity parameters. (FIG. 10B) Co-application of the antiepileptic drugs (AEDs) phenytoin or lamotrigine lead to a dose-dependent decrease in affected, increased network activity measures.

FIG. 11A-11B. Immunofluorescence staining of neuronal and astroglial markers in human neural co-cultures using direct neuronal induced neurons and primary human astroglial cells. (FIG. 11A) Staining of synaptic and pan-neuronal markers in 24-well format and staining of pan-neuronal, neuronal inhibitory, and astroglial markers in human neural co-cultures in 384 well format. (FIG. 11B) Panel of neuronal subtype-specific marker and synaptic marker staining.

FIG. 12A-12B. Readouts of neuronal activity from neural co-cultures with different cell compositions and at different maturation time points. (FIG. 12A) Upper pane: Increase in neuronal activity dependent on total number of neurons and percentage of inhibitory neurons (measured by number of active electrodes), lower panel: response strength to GABA-inhibitor application dependent on total number of neurons and percentage of inhibitory neurons. (FIG. 12B) Upper panel: response strength of neural co-cultures to the GABA-antagonist bicuculline (BIC) at different time points of neuronal network maturation, measured in spiking (mean firing rate, MFR). Lower panel: neuronal network synchronization of in response to BIC at different time points of maturation, measured in synchrony. At 18 days post plating (DPP), BIC application showed significant increase in synchrony. However, at DPP 30, which usually exhibits already highly synchronized network activity, synchrony could not further be increased upon compound dosing.

### DETAILED DESCRIPTION OF THE INVENTION

A flexible, multiplex screening assay is provided for screening biological differences between genetic variations and biological activity classification of biologically active agents and their combinations, including the prediction of neurotoxicity. The data resulting from the assays can be processed to provide robust comparisons between the response of different cells, e.g. differing in genotype, differing in neuronal type, differing in maturity; etc. and agents; for identification of gene-associated phenotypes and classification of agents by their effect on neurons.

The assay methods and compositions of the invention utilize human neural co-cultures of induced neuronal cells and glial cells for screening biologically relevant neuronal and glial function. Human neural co-cultures of the invention may comprise one specific neuronal subtype, mixtures of neuronal subtypes; defined combinations of specific neuronal subtypes, etc., and may comprise without limitation one or multiple defined types of induced neuronal cells such as glutamatergic excitatory, GABAergic inhibitory, dopaminergic, and serotonergic neurons together with human glial cells, including astrocytes, oligodendrocytes and microglia.

The provided human neural co-cultures exhibit complex neuronal functions in vitro, including without limitation spontaneous synchronized network activity, and can be combined with monitoring platforms to a generate a neural screening system. The neural screening system can be used for screening purposes to identify changes in neuronal and glial function caused by chemical agents, genetic agents or culture conditions, as well as to study the effects of genetic variations on neuronal and glial function. The primary readouts for assays using the provided neural screening system are based on the optical or electrical detection of neuronal firing. The main phenotypic assessment includes parameters that describe changes in basic spiking behavior and neuronal network activity.

A feature of the neural co-culture system of the invention is the use of differentiation protocols that allow for development of complex neuronal activity in a short period of time, e.g. after about 2 weeks, about 3 weeks, about 4 weeks, etc. Complex neuronal activity requires the formation of functional circuits and networks evidenced by synchronous firing of neurons in the co-culture system. Synchronous firing is a result of action potentials being propagated via synaptic transmission throughout the neurons connected within a network thereby leading to an avalanche of spiking events that encompasses a large fraction of cells. The co-cultures of the invention can generate synchronous firing from the combination of human induced neuronal cells and glial cells. In some embodiments, the glial cells are human cells. In some embodiments, the glial cells are mouse cells. Suitable methods for induction of neural cells are found, for example, U.S. Pat. No. 9,057,053, herein specifically incorporated by reference.

Before the present methods and compositions are disclosed and described in detail, it is to be understood that this invention is not limited to particular compositions and methods described, as such may, of course, vary. Particularly, specified readout parameters may vary or be expanded depending on specific applications, technical development, or be modified based on knowledge learned by practice of the invention. Methods for generating induced neuronal cells and differentiating glial cells may vary and be refined or adjusted based on progress in the field of neural reprogramming or optimization procedures applying the invention. It is also to be understood that the terminology used herein is for the purpose of describing particular embodiments only and is not intended to be limiting, since the scope of the present invention will be limited only by the appended claims. As used in the specification and the appended claims, the singular forms “a,” “an” and “the” include plural referents unless the context clearly dictates otherwise. Ranges may be expressed herein as from “about” one particular value, and/or to “about” another particular value. Similarly, values may be expressed as approximations, by use of the antecedent “about”.

### DEFINITIONS

Synchronous firing. By synchronous firing, it is intended that a plurality of neurons present in an in vitro culture are functionally connected, such that the majority of the neurons present in the culture, e.g. well, dish, etc., fire at substantially the same time. The number of synchronously firing neurons may be at least about 10, at least about 50, at least about 100, at least about 500, at least about 10³, at least about 5×10³, at least about 10⁴ or more. In some embodiments, the property of synchronous firing in a co-culture system of the invention is facilitated by the fast maturation speed of the neuronal cell component, which is achieved through the method of generating the neurons by direct neuronal cell induction rather than stepwise neuronal differentiation and through co-culturing the generated neurons together with human glial cells.

In the context of an MEA system, detection of action potentials in neural cultures, signals can be detected as spikes when exceeding a present voltage increase, e.g. 2×, 3×, 4×, 5×, 6× or more the standard deviation of average voltages measured by each electrode. A set of sequential spikes may be defined as a burst if at least about 3, about 4, about 5 or more spikes are detected by one electrode within a defined period of time, e.g. from around about 10-500 milliseconds, around about 50 to about 250 milliseconds, or around about 100 milliseconds. Bursts detected across multiple electrodes per well can be defined as synchronized network bursts if the first spikes of individual bursts are co-occurring within about 5, about 10, about 20, about 30, about 40 milliseconds; measured by at least about 20, 25, 30, 35, 40, 45, 50, 55, 60, 65, 70, 75, 80, 85, 90, or 95% of active electrodes.

Firing behavior is influenced by the mixture of specific inhibitory and excitatory neuronal subtypes, the overall density of neurons, the maturation status of the neuronal component, the glial cells, and the ion composition of the culture medium. This particularly applies to the frequencies of firing of individual cells or synchronous firing. Moreover, the percentage of cells being activated as part of synchronized activity patterns (synchronized bursts) of action potentials can also vary. Baseline frequencies of individual and synchronous firing as well as the fraction of neurons being activated during synchronous bursts may be modified by the user through specifically altering parameters, e.g. ion composition of the recording medium, the total amount of neurons per culture, excitatory/inhibitory cell ratios to address specific biological questions of neuronal screening, etc.

The formation of synchronized neuronal network activity represents a high order function of neurobiology as it integrates diverse levels of molecular processes and morphological functionality, including cell-autonomous properties and synaptic transmission. Alterations of synchronous firing that result from exposure to a test agent, genetic effects, or modulated gene expression, etc. provide biologically relevant information about the effects on human neural cells. The readout parameter may include synchronous burst frequency, single burst duration, periodicity of synchronous bursts, duration of synchronous bursts, number of individual spikes per synchronous burst, intervals between synchronous bursts, grouping behavior of synchronous bursts, number of neurons contributing to synchronous bursts, overall synchrony, etc.

Induced neuronal cells. Induced neuronal cells (iN cells) are neuronal cells that have been generated by direct conversion (also referred to as neuronal reprogramming) of cell identity from either somatic cells, somatic stem cells or pluripotent stem cells. In some embodiments, direct conversion of cell identity is achieved by exogenous expression of cell type-specific transcription factors, including without limitation the methods of U.S. Pat. No. 9,057,053, herein specifically incorporated by reference. In some embodiments, direct conversion of cell identity is achieved by activation of endogenous cell type-specific transcription factors, e.g. though application of small molecules or CRIPSR/Cas9-mediated (e.g. regulatory protein domains fused to dCas9) gene regulation. The neuronal identity of the generated cells may be defined, for example, by the presence of pan-neuronal marker proteins such as MAP2, and TUJ-1 and functional features of a typical neurons such as membrane potential, firing spontaneous and evoked action potentials and the presence of synapses competent for signal transmission.

Neuronal cell types can be derived from human embryonic stem cells, human induced pluripotent stem cells, primary differentiated somatic cell types, or primary somatic stem or precursor cells of the central nervous system using direct induction of neuronal cell identity through the delivery of specific transcription factors. Glial cell types including astroglial cells, oligodendroglial cells, and microglial cells can be derived from human embryonic stem cells, human induced pluripotent stem cells, human primary somatic stem or precursor cells of the central nervous system through the delivery of specific transcription factors, stepwise differentiation, targeted activation of endogenous specific transcription factors or through the isolation of mature glial cells from primary neural tissue. Human tissue or cell lines used for generating neural cell types can comprise a complex disease-relevant genotype as being derived from actual patients with a known genetic background or a normal genotype as being derived from healthy individuals or from genetically modified cell lines. For more defined assays conditions, neural cells can be derived from well-established and characterized human induced pluripotent stem cells with complete genome information.

Cell cultures of the invention typically employ homogenous cell populations, or pre-defined ratios of cells, e.g. excitatory neurons, inhibitory neurons, and glial cells. These cell cultures are created by specific culture conditions and cellular manipulation that simulates biologically relevant cellular physiology of the state of interest and to allow for the status of cells in culture to be determined in relation to a change in an environment.

Neuronal cells generated through the direct neuronal induction method (iN cells) include glutamatergic predominantly excitatory neurons, GABAergic predominantly inhibitory neurons, cholinergic neurons, serotonergic neurons, noradrenergic neurons, dopaminergic neurons, and motor neurons. Glutamatergic predominantly excitatory neurons are generated by culture in vitro using the methods set forth in U.S. Pat. No. 9,057,053, herein specifically incorporated by reference, which methods comprise contacting human pluripotent stem cells with any of the following agents, alone or in combination: Neurogenin (Ngn2), NeuroD, Brn2, Ascl, Emx, Fezf2, Cux2, Tbr1, Satb2, Myt1L, and Lhx2. GABAergic predominantly inhibitory neurons are generated by contacting human pluripotent stem cells with any of the following agents, alone or in combination: Ascl, Dlx1/2/5, Myt1L, Nkx2.1, Lhx6/8, Sox2, Foxg1 and Ctip2. Cholinergic neurons are generated by contacting human pluripotent stem cells by any of the following agents, alone or in combination: Neurogenin (Ngn2), NeuroD, Ascl, Brn2, Lhx3, Hb9, Isl1, Isl2, Brn3a, Fezf2, and Klf7. Serotonergic are generated by contacting human pluripotent stem cells with any of the following agents, alone or in combination: Ascl, Neurogenin (Ngn2), Gata2/3, FoxA2, Lmx1a/b, Nkx2.2, Ptx, and FEV. Noradrenergic neurons are generated by contacting human pluripotent stem cells with any of the following agents, alone or in combination: Ascl, Neurogenin (Ngn2), HMX1, Phox2a/b, Hand2, and Gata2/3. Dopaminergic neurons are generated by contacting human pluripotent stem cells by any of the following agents, alone or in combination: Ascl, Brn2, Lmx1a/b, Neurogenin (Ngn2), NeuroD, FoxA2, FEV, Otx2, Nurr1, Pitx3, and En1. Motor neurons are generated by contacting human pluripotent stem cells by any of the following agents, alone or in combination: Neurogenin (Ngn2), NeuroD, Ascl, Brn2, Lhx3, Hb9, Isl1, Isl2, Brn3a, and Fezf2. Yang et al. (2017) Nat Methods 14(6):621-628 describes the generation of GABAergic inhibitory neurons from human pluripotent stem cells, incorporated by reference.

Glial cells. In order to develop the degree of synaptic competence required to form synchronized network activity the generated neurons are co-cultured with human or animal-derived glial cells starting day 5 after neuronal induction until accomplishment of neuronal activity recording (up to 20 weeks). The types of human glial cells used in this method include astroglial cells (or astrocytes), oligodendroglial cells (or oligodendrocytes), and microglial cell as well as various related derivatives, subtypes, activation states, and maturation levels. Here, astroglial type of cells are defined as cells that have attributes such as high rates of glutamate uptake, stain positive for ALDH1L1, GFAP, S100β or CD44, release the proteins ApoE, GDNF, Thrombospondin, and support synaptogenesis and neuronal maturation. Oligodendroglial type of cells are defined as cells that stain for O4, MBP, or OLIG2 and interact with co-cultured neurons to enhance viability and influence electrophysiological properties. Microglial type of cells are defined as cells that can secrete cytokines, are capable of phagocytosis and antigen-presentation, and stain positive for CD11b, CD45, IBA1 or PU.1. Astroglial cells can be isolated from human brain tissue or be generated from human primary neural stem cells (NSCs) by contacting the cells with any of the following agents, alone or in combination: whole serum, single serum components, insulin, CNTF, BMP2/4, NFIA, NFIB, SOX9, and HES1. Furthermore, astroglial cells can be generated from human pluripotent stem cells through either stepwise differentiating the cells towards a neuroepithelial and subsequently astroglial identity by withdrawal of BMP and TGFβ an subsequent culturing in neural medium supplemented with of CNTF and EGF or through directly reprogramming human pluripotent stem cells into the astroglial lineage using transcription factors like NFIA, NFIB, SOX9, HES1, alone or in combination. Oligodendroglial cells Microglial cells can be isolated from human or animal brain tissue or be generated from human pluripotent stem cells through stepwise differentiating towards an neuroepithelial and subsequently oligodendroglial identity. This may be accomplished by initial inhibition of BMP and TGFβ signaling and exposure to retinoic acid, followed by activation of sonic hedgehog signaling and culturing in neural medium. Subsequently, media is supplemented with oligodendroglial lineage supporting growth factors and agents such as PDGF, IGF, insulin, NT3, cAMP, T3, and HGF. Lineage commitment and oligodendroglial precursor expansion is then followed by growth factor withdrawal and final maturation of oligodendrocytes on adequate substrate such as PO/laminin or matrigel. Oligodendroglial specification of human pluripotent stem cells, neural stem cells or early glial progenitor cells can further be enhanced through exogenous overexpression of Olig2. Microglial cells can be isolated from human or animal brain tissue or be generated from human pluripotent stem cells through stepwise differentiating towards an endothelial and subsequently a yolk sac myeloid and finally microglial (macrophage-like) identity. This may be accomplished by initial inhibition of BMP and TGFβ signaling followed by culturing in neuroglial differentiation media with timed exposure to G-CSF/GM-CSF/CSF1, IL-34, SCF, IL-3, IL-6, BMP4, Activin A, and Flt3L.

Genetically modified cells. Neural cells that have been genetically altered, e.g. by transfection or transduction with recombinant genes or by antisense technology, or by using the CRISPR/Cas9 technology to provide a gain, a loss, or a change of genetic function, may be utilized with the invention. Methods for generating genetically modified cells are known in the art, see for example “Current Protocols in Molecular Biology”, Ausubel et al., eds, John Wiley & Sons, New York, N.Y., 2000 and “Genome Editing in Human Stem Cells”, Byrne et al., Methods Enzymol. 2014. The genetic alteration may be a knock-out, usually where non-homologous end joining DNA repair results in a deletion that knocks out expression of a targeted gene; or a knock-in, where a genetic sequence not normally present in the cell is stably introduced by homology dependent DNA repair; or an introduction of a genetic variation or mutation by replacing a short endogenous DNA sequence with donor DNA. A variety of methods may be used in the present invention to achieve a knock-out, including site-specific recombination, expression of anti-sense or dominant negative mutations, CRISPR/Cas9-mediated targeting and the like. Knockouts have a partial or complete loss of function in one or both alleles of the endogenous gene in the case of gene targeting. Preferably, expression of the targeted gene product is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences are ultimately deleted from the genome, leaving a net change to the native sequence. Further may neuronal or glial cells with modulated gene expression be used with the invention. Increased gene expression may be achieved by delivering RNA or DNA that carry a reading frame of a specific gene to the cells using transfection methods or viral transduction (e.g. lentivirus, adeno-associated virus, sendai virus, or retrovirus). Decreased gene expression may be achieved by delivering short hairpin RNAs or microRNAs either directly or encoded on DNA constructs, or modified antisense oligonucleotides. Delivery can be achieved using transfection methods or viral transduction.

In addition, cells may be environmentally induced variants of single cell lines: e.g., a responsive cell line split into independent cultures and grown under distinct conditions, for example with or without NGF, in the presence or absence of other growth factors or combinations thereof. Each culture condition then induces specific distinctive changes in the cells, such that their subsequent responses to an environment change is distinct.

The term “environment,” or “culture condition” encompasses the presence of an agent being tested, cells, media, factors, time and temperature. Environments may also include drugs and other compounds, particular atmospheric conditions, pH, salt composition, minerals, etc. The conditions will be controlled and the dataset will reflect the similarities and differences between each of the assay combinations involving a different environment or culture condition.

Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free.

Parameters. The term parameter refers to quantifiable components of cells, particularly components that can be accurately measured, desirably in a medium-to-high throughput system. A parameter can be any (multi)cellular process including changes in membrane potentials as well as intra- and extra cellular ion concentrations, cell component, or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. In some embodiments a parameter is Ca⁺⁺ release. In some embodiments, a parameter is measuring electric current or potentials as a result of neuronal membrane depolarization. In some embodiments a parameter is measuring characteristics of cellular morphology, viability, cellular trafficking, morphology of organelles, localization of organelles, trafficking of organelles, trafficking of lysosomes, function of specific enzymes, or chemical changes in the cytoplasm.

While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assay combinations, usually at least about 2 of the same assay combination will be performed to provide a value. Variability is expected and a range of values for each of the set of test parameters will be obtained using standard statistical methods with a common statistical method used to provide single values.

Markers are selected to serve as parameters based on the following criteria, where any parameter need not have all of the criteria: the parameter is modulated in the physiological condition that one is simulating with the assay combination; the parameter has a robust response that can be easily detected and differentiated and is not too sensitive to concentration variation, that is, it will not substantially differ in its response to an over two-fold change; the parameter is a readily measurable component; the parameter is not co-regulated with another parameter, so as to be redundant in the information provided; and in some instances, changes in the parameter are indicative of toxicity leading to cell death. The set of parameters selected may be sufficiently large to allow distinction between reference patterns, while sufficiently selective to fulfill computational requirements.

For any specific combination of cells, environment and test agent, certain parameters will be functionally relevant and will be altered in response to test or reference agents or conditions, while other parameters may remain static in that particular combination. The dataset may comprise data from at least 1 functionally relevant parameters, at least about 2 functionally relevant parameters, and may include 3 or more functionally relevant parameters. In analyzing the data, not all of the parameters need not be weighed equally. Those parameters that are closely functionally associated with the disease state or pathophysiologic response, and/or with modulation of cell pathways of interest may be given greater weight in evaluating a candidate drug or a readout, as compared to other parameters that are suggestive, but do not have as strong an association.

Parameters of interest include detection of cytoplasmic, cell surface or secreted biomolecules, frequently biopolymers, e.g. polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins or aggregated proteins. A parameter may be detection of a specifically cleaved, modified, misfolded, or aggregated protein or oligosaccharide, e.g. a phosphorylated protein, such as a STAT transcriptional protein; or sulfated oligosaccharide, or such as the carbohydrate structure Sialyl Lewis x, a selectin ligand. The presence of the active conformation of a receptor may comprise one parameter while an inactive conformation of a receptor may comprise another.

Parameters of interest may also include morphological changes such as dendrite arborization, axon elongation, density, size and distribution of synaptic puncta, as well as molecular composition and positioning of the axion initial segment. Parameters of interest may also include changes in membrane potential, membrane resistance, and cellular influx/efflux of ions as well as membrane permeability. A parameter may be the quantitative detection of a specific ion, e.g. intracellular Ca²⁺, metabolites, e.g. ATP or ADP, oxidative state of detoxifying molecules, e.g. glutathione and glutathione disulfide, subcellular structures, e.g. assembly of LC3-containing autophagosomes, chemically reactive molecules, e.g. reactive oxygen species, modification of DNA, e.g. chromatin modification, DNA-methylation, DNA damage foci, e.g. gamma-H2AX, and metabolite precursors and intermediate products, e.g. DOPAL. Parameters of interest may also include cellular trafficking such as axonal transport and trafficking of lysosomes, vesicles, and larger organelles. This may also include morphology, distribution, and number of organelles and vesicles. Parameters of interest may further include secretion of exosomes and vesicles as well as extracellular aggregation and clearance of proteins and polypeptides. Parameters may also include the measurement of cell-to-cell interactions e.g. myelination of neurons, demyelination of neurons, pruning of synapses, phagocytosis, induced lysis, apoptosis induction, formation of tight junction, synaptogenesis, etc.

Neuronal activity parameters. Of particular interest for the disclosed neuronal screening system are parameters related to the electrical properties and signal transmission characteristics of the cells and therefore directly informative about neuronal function and activity. Methods to measure neuronal activity may sense the occurrence of action potentials (spikes). The characteristics of the occurrence of a single spike or multiple spikes either in timely clustered groups (bursts) or distributed over longer time (spike train) of a single neuron or a group of neurons indicate neuronal activation patterns and thus reflect functional neuronal properties, which can be described by multiple parameters. Such parameters can be used to quantify and describe changes in neuronal activity in the systems of the invention.

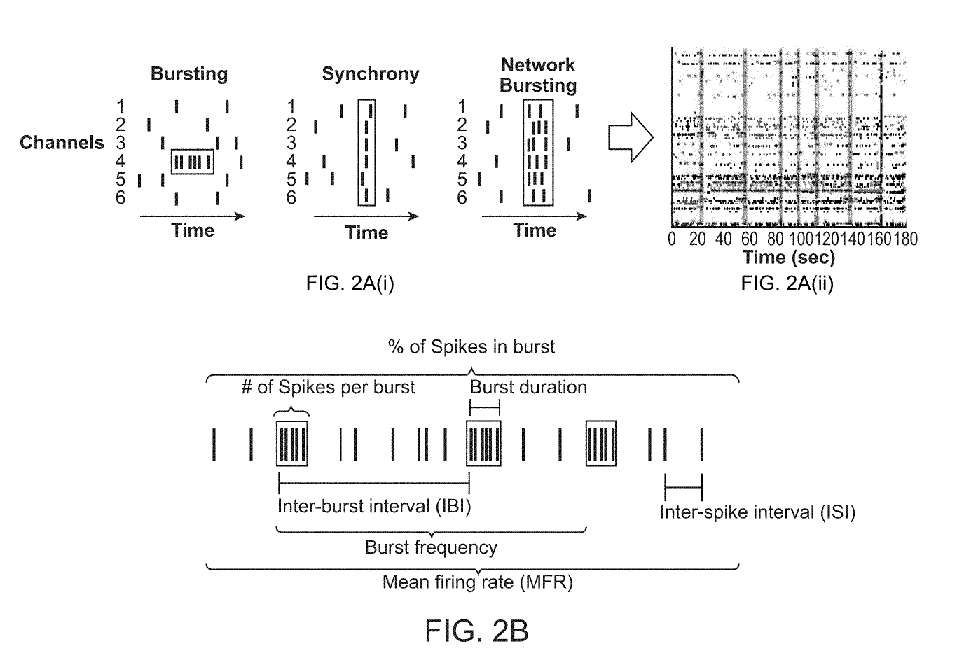

Neuronal activity parameters include, without limitation, total number of spikes (per recording period); mean firing rate (of spikes); inter-spike interval (distance between sequential spikes); total number of bursts (per recording period); burst frequency; number of spikes per burst; burst duration (in milliseconds); inter-burst interval (distance between sequential bursts); burst percentage (the portion of spikes occurring within a burst); total number of network bursts (spontaneous synchronized network activity); network burst frequency; number of spikes per network burst; network burst duration; inter-network-burst interval; inter-spike interval within network bursts; network burst percentage (the portion of bursts occurring within a network burst); cross-correlation of detected spikes between all electrodes per well (e.g. for MEA recordings, measure of synchrony, see FIG. 2B).

Quantitative readouts of neuronal activity parameters may include baseline measurements in the absence of agents or a pre-defined genetic control condition and test measurements in the presence of a single or multiple agents or a genetic test condition in the presence or absence of a candidate agent. Quantitative readouts may include solvent control measurements. Furthermore, quantitative readouts of neuronal activity parameters may include long-term recordings and may therefore be used as a function of time (change of parameter value). Quantitative readout may further be acquired at multiple time points for a neural co-culture to measure latent effects, delayed effects, or long-term effects. Readouts may be acquired either spontaneously or in response to or presence of stimulation or perturbation of the complete neuronal network or selected components of the network. The quantitative readouts of neuronal activity parameters may further include a single determined value, the mean or median values of parallel, subsequent or replicate measurements, the variance of the measurements, various normalizations, the cross-correlation between parallel measurements, etc. and every statistic used to a calculate a meaningful and informative factor.

### Neural Cells and Co-Cultures

The methods and cells described below illustrate the development and use of a human neural co-culture system, which can be combined with a monitoring device, e.g. a multielectrode array platform, for biologically relevant screening of changes in neuronal activity.

Glial cells. For the generation of astroglial and oligodendroglial cells from human pluripotent stem cells (hPSCs) a step-wise differentiation protocol through a transient neuroepithelial cell stage is applied. In alternative embodiments astroglial and oligodendroglial cells are directly differentiated from human neural stem cells. For the differentiation of microglial cells from human pluripotent stem cells a step-wise differentiation protocol through a transient endothelial cell stage is applied. Once differentiated, the astroglial, oligodendroglial, and microglial cells may be combined with other neural cells in a culture system of the invention. Alternatively, primary glial cells, e.g. mouse glial cells, can be obtained from dissociation of brain tissue.

For the derivation from primary human neural stem cells (NSCs), the stem cells are differentiated by culture in neural media in the presence of an effective dose of EGF and serum or BMP2/4 for a period of time sufficient to expand astroglial cells.

For derivation from human pluripotent stem cells, hPSC colonies are detached as clumps and cultured in bFGF-free human embryonic stem cell medium (hES medium, DMEM/F12 (containing L-Glutamine and Sodium bicarbonate)+20% KSR+Glutamax [2 mM]+NEAA [100 μM]+2-mercaptoethanol [100 μM]+sodium pyruvate) in the presence of an effective dose of a ROCK inhibitor and effective doses of SMAD signaling inhibitors to generate embryoid bodies. These embryoid bodies are then seeded in neural medium on PO/laminin-coated plates to form neuroepithelial cells. Neuroepithelial cells are then detached and cultured in neural medium to form neurospheres. The neurospheres are resuspended in medium with an effective dose of EGF and bFGF to generate astroglial committed spheres which can be resuspended as single cells in neural medium with serum or BMP2/4 and an effective dose of CTNF.

Specific steps in differentiation of astroglial cells may comprise, for example, the following: hPSC colonies cultured on mouse feeder cells (SNL 76/7) or under feeder-free conditions on matrigel are detached using dispase enzyme at 37° C. Detached hPSC colnies cells are washed once with pre-warmed DMEM/F12 medium and pelleted by gravity. Subsequently, clumping hPSC colonies are carefully resuspended in mouse feeder-cell-conditioned human embryonic stem cell medium (hES medium, DMEM/F12 (containing L-Glutamine and Sodium bicarbonate)+20% KSR+Glutamax [2 mM]+NEAA [100 μm]+2-mercaptoethanol [100 μm]+Sodium Pyruvate) containing 10 μm Rock inhibitor (Y27632) and dual SMAD inhibitors (e.g. 10 μm SB431542 and 250 nM LDN193189) and cultured on low-attachment plates to form embryoid bodies. This medium is changed daily until day 4 (d4) after differentiation induction when the medium is replaced by ¾ of hES medium without bFGF containing inhibitors of SMAD signaling and ¼ N2 medium (N2 medium, 500 ml DMEM/F12, 5 ml N2 supplement, ½ B27 supplement, 5 ml MEM non-essential amino acids, 5 ml Glutamax, 1×β-mercaptoethanol). At day 6 the medium is replaced by ½ of hES medium without bFGF containing inhibitors of SMAD signaling and ½ N2 medium. At day 7 the medium is replaced by N2 medium and the embryoid bodies are seeded on PO/laminin-coated plates. At day 8 N2 medium ich changed completely and optionally supplemented with 0.5 μm retinoic acid or 100-500 ng/ml SHH for caudalization or ventralization, respectively. Afterwards, half media changes are performed every other day using N2 medium. At day 12 after differentiation induction between 3-10 neural rosettes are observed within each of the forming neuroepithelial cell colonies. Colonies that exhibit a flattened morphology without signs of rosette formation are removed and the remaining colonies are lifted mechanically. The harvested neuroepithelial colonies are pelleted by centrifugation for 2 minutes at 100×g and the supernatant is discarded. Pelleted neuroepithelial colonies are resuspended in neural medium (500 ml DMEM/F12, 5 ml N2 supplement, 5 ml MEM non-essential amino acids, 1 ml heparin [1 mg/ml]) and transferred to an uncoated flask. At day 14 and day 16 medium changes are performed by replacing ⅔ of the old medium by fresh N2 medium. At day 19 forming spheres are pelleted by centrifugation for 2 minutes at 100×g and the supernatant is discarded. The pelleted spheres are resuspended in neural medium supplemented with 10 ng/ml EGF and 10 ng/ml bFGF and transferred to a new uncoated flask. At day 21 the flask is tilted to allow spheres to sink by gravity and ⅔ of the medium is replaced by fresh neural medium supplemented with 10 ng/ml EGF and 10 ng/ml bFGF. A flamed and curved Pasteur pipette with an opening diameter between 0.3 and 0.5 mm is used to break down large spheres by pipetting up and down. This procedure is repeated every 3 days until day 90 after differentiation induction when the astroglial committed spheres are pelleted by centrifugation for 2 minutes at 100×g. The supernatant is carefully aspirated and the spheres are washed once with 0.5 mM EDTA in PBS. Subsequently, the spheres are dissociate by 5 minutes incubation with accutase enzyme mix at 37° C. Afterwards, the dissociated astroglial progenitors are washed twice with prewarmed DMEM/F12, pelleted by centrifugation for 2 minutes at 100×g, and resuspended as single cells in neural medium containing 5% fetal bovine serum (FBS) and 10 ng/ml CTNF. The resuspended astroglial progenitors are then seeded at 10,000 cells/cm² on matrigel coated plates. In the following, the medium (neural medium containing 5% FBS and 10 ng/ml CTNF) is change completely every 3 days until day 100 when the mature astrocytes are ready to be replated for neural cocultures.

In some embodiments, for the generation of astroglial cells from primary human neural stem cells (NSCs) a direct differentiation protocol is applied. Briefly, NSCs are expanded in neural stem medium (neural stem medium: 250 ml Neurobasal-A medium, 250 ml DMEM/F12 medium, 10 mM HEPES, 1 mM sodium pyruvate, 100 μm non-essential amino acid solution, 2 mM Glutamax, 1×B27 supplement without vitamin A, 1×N2 supplement, 20 ng/ml EGF, 20 ng/ml FGF, 10 ng/ml human LIF, and 2 μg/ml heparin). For differentiation, NSCs are dissociated using accutase enzyme mix and plated in neural medium (neural medium: 500 ml DMEM/F12, 5 ml N2 supplement, 5 ml MEM non-essential amino acids, 1 ml heparin [1 mg/ml]) supplemented with 10 ng/ml EGF and 3% fetal bovine serum on matrigel-coated 10 cm tissue culture dishes. Complete medium changes are performed twice a week and astroglial cells are expanded for at least 3 weeks under the same conditions before being used for neural co-cultures.

For the derivation from primary human neural stem cells (NSCs), the stem cells are differentiated by culture in neural media (neural medium: DMEM/F12 medium, 1 mM sodium pyruvate, 100 μm non-essential amino acid solution, 2 mM Glutamax, B27 supplement without vitamin A, and N2 supplement) in the presence of effective doses of retinoic acid and a sonic hedgehog signaling pathway agonist, e.g. purmorphamine, followed by exposure of the cells to effective doses of PDGF, NT3, insulin, IGF, biotin and HGF for a period of time sufficient to expand oligodendroglial cells.

For derivation from human pluripotent stem cells, hPSC colonies are detached as clumps and cultured in bFGF-free human embryonic stem cell medium (hES medium, DMEM/F12 (containing L-Glutamine and Sodium bicarbonate)+20% KSR+Glutamax [2 mM]+NEAA [100 μm]+2-mercaptoethanol [100 μm]+sodium pyruvate) in the presence of an effective dose of a ROCK inhibitor, effective doses of BMP and TGFβ signaling inhibitors, and effective doses of sonic hedgehog signaling and wnt signaling agonists to generate embryoid bodies. After 4 days, embryoid bodies are seeded on matrigel-coated plates in neural medium in the presence of effective doses of BMP and TGFβ signaling inhibitors and effective doses of sonic hedgehog signaling and wnt signaling agonists to generate neuroepithelial cells. Emerging neuroepithelial cells are further cultured in neural medium containing effective doses of sonic hedgehog signaling and wnt signaling agonists and ascorbic acid, followed by application of effective doses of bFGF to generate neural stem cells. Neural stem cells are expanded in neural medium in the presence of effective doses of EGF, bFGF and human LIF. Expanded neural stem cells are then cultured in neural media in the presence of effective doses of retinoic acid and a sonic hedgehog signaling pathway agonist, e.g. purmorphamine, followed by exposure of the cells to effective doses of PDGF, NT3, insulin, IGF, biotin and HGF for a period of time sufficient to expand oligodendroglial cells.

For derivation of microglial cells from human pluripotent stem cells, the cells are disaggregated and initially cultured in human embryonic stem cell medium the presence of an effective dose of a ROCK inhibitor. Differentiation is induced by culturing in human embryonic stem cell medium in the presence of effective doses of bFGF, BMP4, Activin A and LiCl under hypoxic conditions. After 2 days, media is replaced by serum-free neuroglial differentiation media (Neurobasal-A medium, 2.3 μg/l BSA, 50 mM NaCl, 10 mM HEPES, 1 mM sodium pyruvate, 100 μm non-essential amino acid solution, 2 mM Glutamax, B27 supplement, and N2 supplement) in the presence of effective doses of bFGF and VEGF under hypoxic conditions. After 4 days, media is replaced by serum-free neuroglial differentiation media containing effective doses of bFGF, VEGF, TPO, SCF, IL3, and IL6 and the cells are cultured under normoxic conditions from there on. After 10 days, CD43+ cells are isolated and reseeded in serum-free neuroglial differentiation media containing effective doses of MCSF, IL3, TPO, SCF1, FLT3, IL34, and TGFβ1. After 14 days, media is replaced by serum-free neuroglial differentiation media containing effective doses of MCSF, CSF1, FLT3, IL34, TGFβ1, and insulin. After 25 days, media is replaced by serum-free neuroglial differentiation media containing effective doses of MCSF, CSF1, FLT3, IL34, TGFβ1, insulin, CD200, and CX3CL1 for expansion. After 35-40 days, cells are reseeded on primary astroglial cells in neuroglial differentiation media containing effective doses of CD200 and CX3CL1 for microglial maturation.

Excitatory neurons. For the generation of excitatory neurons cells from human pluripotent stem cells (hPSCs) a direct differentiation protocol through exogenous expression of neurogenic transcription factors may be used. The hPSC are cultured in the presence of medium and an effective dose of a ROCK inhibitor, and induced to express an effective dose of Ngn2 or NeuroD1, e.g. by lentiviral infection. The cells are cultured, e.g. in neuronal medium, in the presence of an effective dose of a ROCK inhibitor until neuronal differentiation initiates to generate committed immature induced neuronal cells, which can be replated in medium for the neural co-cultures.

Specific steps in differentiation of excitatory neurons may comprise, for example, the following: the transcription factor Ngn2 is co-expressed with a resistance gene against puromycin, for the purpose of selection, using lentiviral transduction. In detail, hPSCs are grown on gelatin-coated 6-well plates on mouse feeder cells (SNL 76/7) and expanded until nearly reaching confluency. Alternatively, hPSCs can also be expanded and maintained under feeder-free condition using matrigel surface coating and mTeSR1 stem cell medium. At the day of viral infection (day-1) conditioned human embryonic stem cell medium (hES medium, DMEM/F12 (containing L-Glutamine and sodium bicarbonate)+20% KSR+Glutamax [2 mM]+NEAA [100 μm]+2-mercaptoethanol [100 μm]+sodium pyruvate+bFGF [10 ng/ml]) is prepared by adding 2 ml of medium to each well of a 6-well plate with mouse feeder cells and incubating the medium for at least 4 hours at 37° C. and 5% CO₂. Meanwhile a 6-well plate is coated with a 1:100 dilution of matrigel and incubated at 37° C. for at least one hour. Prior to harvesting hPSCs the mouse feeder cells are removed by washing twice with PBS and adding 350 μl CTK enzyme mix (CTK: 5 ml of 2.5% Trypsin+5 ml of 1 mg/ml collagenase IV+0.5 ml of 0.1M CaCl₂+10 ml KSR, 30 ml ddH₂O) to each well of a 6-well plate. The cells are incubated for 3 minutes at 37° C. until the feeder cells start to come off. CTK is aspirated and the remaining hPSCs are washed twice with 0.5 mM in PBS. Subsequently, 1 ml accutase enzyme mix is added to each well and the cells are incubated for another 3 minutes at 37° C. The dissociating cells are collected in 4 ml prewarmed DMEM/F12 per well and pelleted by centrifugation for 5 minutes at 200×g. The harvested hPSCs are then resuspended in conditioned hES medium (or mTeSRmedium) containing 10 μm Rock inhibitor (Y27632) and the cell number is adjusted to 1×10⁵-1.75×10⁵ cells/ml. For efficient lentiviral infection between 2.5 and 3.5 μl of 100-fold concentrated virus per ml is added to the suspension including lentivirus carrying an Ngn2-T2A-puro expression construct under a tet-on promoter and lentivirus coding for rtTA (reverse tetracycline transactivator). The suspension is mixed carefully by pipetting up and down and 2 ml are seeded per well of the 6-well plate coated earlier with matrigel. After 12-24 hours (day 0) the cells are induced by removing 1 ml of the hES medium and adding 1 ml N3 medium (N3 medium: DMEM/F12+1×N2 supplement+B27 supplement+Insulin [10 μg/ml]+1×NEAA) containing 2 μg/ml doxycycline and 10 μm Rock inhibitor (Y27632). At day 1 the medium is aspirated completely and replaced by N3 medium containing 2 μg/ml doxycycline and 2 μg/ml puromycin. At day 2 the medium is changed completely and first bipolar extension become visible at the cells. At day 3 the medium is aspirated completely and replaced by N3 medium containing 2 μg/ml doxycycline and 2 μg/ml puromycin and 2 μm arabinofuranosyl cytosine (AraC). At day 4 the committed and non-proliferative immature induced neuronal (iN) cells are harvested by washing once with 0.5 mM EDTA in PBS and 5 minutes incubation with accutase enzyme mix at 37° C. Dissociated immature iN cells are then collected in prewarmed DMEM/F12 and pelleted by centrifugation for 5 minutes at 300×g. Pelleted iN cells are then resuspended in neurobasal/B27 medium (Neurobasal/B27 medium: Neurobasal-A medium+B27+0.5×Glutamax+NT3 [10 ng/ml]+mouse laminin [200 ng/ml]+doxycycline [2 μg/ml]+1% FBS) containing 10 μm Rock inhibitor (Y27632) and are ready to be replated for neural co-cultures.

Inhibitory neurons. For the generation of excitatory neurons cells from human pluripotent stem cells (hPSCs) a direct differentiation protocol through exogenous expression of neurogenic transcription factors may be used. The hPSC are cultured in the presence of medium and an effective dose of a ROCK inhibitor, and induced to express an effective dose of Ascl1, Dlx2, and Myt1L, e.g. by lentiviral infection. The cells are cultured, e.g. in N3 medium, in the presence of an effective dose of a ROCK inhibitor until neuronal differentiation initiates to generate committed immature induced neuronal cells, which can be replated in medium for the neural co-cultures.

Specific steps in differentiation of inhibitory neurons may comprise, for example, the following: using exogenous expression of neurogenic transcription factors e.g. by lentiviral transduction. Briefly, hPSCs are expanded on gelatin-coated 6-well plates on mouse feeder cells using hES medium or expanded feeder-free on matrigel-coated plates using mTeSR1 stem cell medium. At the day of viral infection (day-1) conditioned hES medium is prepared and a 6-well plate is coated with a 1:100 dilution of matrigel. For harvesting the hPSCs, the mouse feeder cells are removed according to the procedure described above, and the remaining hPSCs are dissociated with 1 ml accutase enzyme mix. The dissociating cells are collected, pelleted, and resuspended in conditioned hES medium (or mTeSR1) containing 10 μm Rock inhibitor (Y27632) and the cell number is adjusted to 1.5×10⁵-2×10⁵ cells/ml. For efficient lentiviral infection between 3 and 6 μl of 100-fold concentrated virus per ml and construct is added to the suspension. Lentiviruses for transduction include constructs for Ascl1-T2A-puro, Dlx2-T2A-hygro, and Myt1L expression under a tet-on promoter as well as lentivirus coding for rtTA. The suspension is mixed and 2 ml are seeded per well of the 6-well plate pre-coated with matrigel. At day 0, the cells are induced by removing 1 ml of the hES medium and adding 1 ml N3 medium (N3 medium: DMEM/F12+1×N2 supplement+B27 supplement+Insulin [10 μg/ml]+1×NEAA) containing 2 μg/ml doxycycline and 10 μm Rock inhibitor (Y27632). At day 1, the medium is aspirated completely and replaced by N3 medium containing 2 μg/ml doxycycline, 2 μg/ml puromycin, and 100 μg/ml hygromycin. At day 2, the medium is changed completely. At day 3 and 4, half of the medium is changed. At day 5, the medium is aspirated completely and by N3 medium containing 2 μg/ml doxycycline, 100 μg/ml hygromycin, and 2 μm AraC. At day 6, immature induced neuronal (iN) cells are harvested using accutase enzyme mix. Dissociated immature iN cells are then collected, pelleted, and re-suspended in neurobasal/B27 medium containing 10 μm Rock inhibitor (Y27632) for seeding for neural co-cultures.

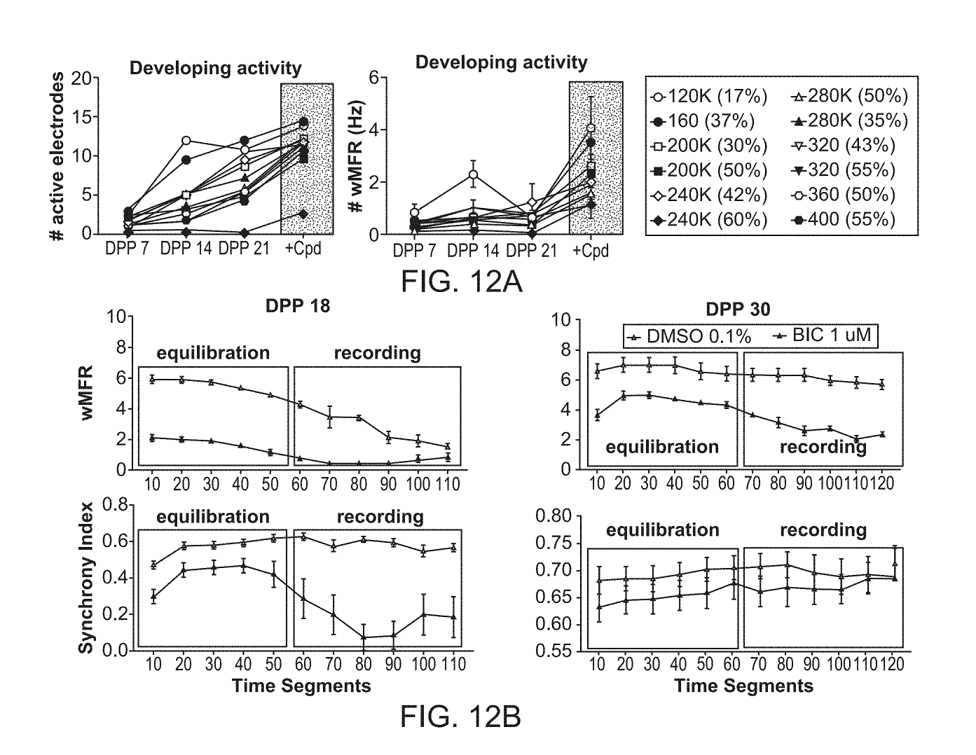

Neural Co-cultures. One or more of the neuronal subtypes described above can be provided in a co-culture of the invention. In some embodiments, glial cells and one or both of excitatory and inhibitory neurons are present. The cells derived as discussed above can be plated for the desired combination. The ratio of excitatory/inhibitory neurons may be around about 90:10; 80:20; 70:30; 60:40; 50:50, 40:60; 30:70; 20:80; 10:90; etc. In some embodiments, the percentage of excitatory neurons in the combined excitatory/inhibitory neurons is about 10%, 20%, 30%, 40%, 50%, 60%, 70%, 80%, or 90%. In some embodiments, the percentage of excitatory neurons in the combined excitatory/inhibitory neurons is from about 10% to about 20%, 30%, 40%, 50%, 60%, 70%, 80% or 90%, from about 20% to about 30%, 40%, 50%, 60%, 70%, 80% or 90%, from about 30% to about 40%, 50%, 60%, 70%, 80% or 90%, from about 40% to about 50%, 60%, 70%, 80% or 90%, from about 50% to about 60%, 70%, 80% or 90%, from about 60% to about 70%, 80% or 90%, or from about 70% to about 80% or 90%, all inclusive (FIG. 12A). Normally glial cells are present, where the ratio of glial cells to neuronal cells is around about 1:10, 1:7.5, 1:5, 1:2.5, 1:1; etc. In some embodiments, the percentage of glia cells to the combined glia/neuronal cells is neurons is about 10%, 15% 20%, 25%, 30%, 35%, 40%, 45%, 50%, 55%, 60%, 65%, 70%, 80%, or 90%. In some embodiments, the percentage of glia cells to the combined glia/neuronal cells is neurons is from about 10% to about 15%, 20%, 25%, 30%, 35%, 40%, 45%, 50%, 60%, 70%, 80% or 90%, from about 20% to about 25%, 30%, 35%, 40%, 45%, 50%, 60%, 70%, 80% or 90%, from about 30% to about 35%, 40%, 45%, 50%, 60%, 70%, 80% or 90%, from about 40% to about 50%, 60%, 70%, 80% or 90%, from about 50% to about 60%, 70%, 80% or 90%, from about 60% to about 70%, 80% or 90%, or from about 70% to about 80% or 90%, all inclusive. The number of neurons plated may be from about 10⁴, 10⁵, 10⁶ per well or more.

In some embodiments the provided neuronal screening systems comprise specific defined combinations of neurons and glial cells, e.g. the ratio of neuron to glial cells may be about 1:10, 1:5, 1:3, 1:2, 1:1, 2:1, 3:1; 5:1, 10:1 and the like. The neuron component may comprise defined ratios of different neurons, e.g. inhibitory and excitatory, at a ratio of from about 1:20, 1:15, 1:10, 1:5, 1:3, 1:2, 1:1, 2:1, 3:1, 5:1, 10:1, 15:1, 20:1, and the like, Neurons may comprise any of the previously described classes. The specific ratio may be determined by the intention of the assay, e.g. to simulate Parkinson's disease, ADHD, Alzheimer's disease, etc.

For example, a 1:2 ratio of GABAergic inhibitory neurons to glutamatergic excitatory neurons in the presence of a 30% fraction of astroglial cells may be used; etc. Similarly, the ratio of inhibitory to excitatory neurons can be increased to e.g. 1:1 in order to study effects of agents or genotypes that particularly influence the inhibitory component of a neural network, like seizure-inducing compounds. Consequentially, the provided neuronal screening system can be used to identify agents to treat epilepsy and seizure disorders, e.g. caused by mutations in the SCN1A or GABRG2 genes, or antagonize compound-induced neuronal convulsion. Moreover, the screening assay can be expanded to include different ratios of GABAergic, glutamatergic and dopaminergic neurons in order to study the effects of agents and genotypes on diseases marked by a disturbed dopamine homeostasis, like Parkinson's disease (PD), depression, and attention deficit hyperactivity disorder (ADHD).

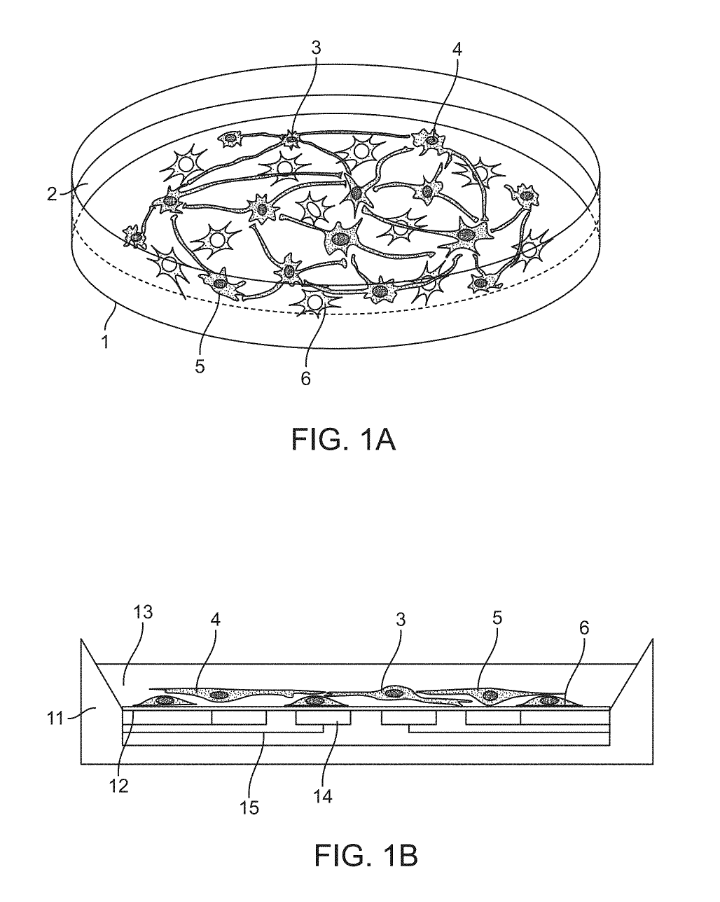

The different cell types of the systems are combined according to the desired phenotypic readout of the application, e.g. modulating effects of compounds on inhibitory neurons in a neuronal network (seizure assays). The neural co-culture system may be of a size appropriate for the assay, typically comprising up to about 5×10⁴, up to about 10⁵, up to about 5×10⁵, about 10⁶, up to about 5×10⁶ neurons, up to about 10⁷ neurons. The neural co-culture may comprise up to about 5×10⁴, up to about 10⁵, up to about 2.5×10⁵, about 5×10⁵ glial cells. The neural co-culture system is grown on a suitable adhesive substrate depending on the detection method used for measuring neuronal activity (FIG. 1A, element 1). Media composition for neural co-culture system may vary in ion content, nutrient, and growth/specification factor supplementation according to applied detection method (FIG. 1A, element 2).

In some embodiments, the neural cells are seeded and maintained on MEA plates, which are specialized tissue culture plates comprising microelectrodes integrated into the well bottom for detection of extracellular currents and local field potentials (see, for example, the Maestro Platform from Axion BioSystems). The MEA plated may be precoated with a suitable substrate, including without limitation laminin, PEI, matrigel, etc.

Specific plating steps may comprise, for example the following steps: Before replating the iN cells and glial cells, the MEA plates are pre-coated with matrigel (12-well format, glass surface) or polyethylenimine (PEI) and laminin (48-well and 96-well formats, plastic surface). Pure excitatory, pure inhibitory, or a mixture of excitatory and inhibitory iN cells are seeded at different densities depending on the type of assay, for example to reach a final ratio excitatory/inhibitory iN cells of 70%/30% for modeling physiological conditions. Neuronal cells are plated in neurobasal/B27 medium (Neurobasal/B27 medium: Neurobasal-A medium+B27+0.5×Glutamax+NT3 [10 ng/ml]+mouse laminin [200 ng/ml]) supplemented with 2 μg/ml doxycycline, 1% FBS, and 10 μm Rock inhibitor (Y27632). The total number of seeded iN cells may be in the range between 300,000 and 600,000 cells per well for 12-well or between 100,000 and 250,000 cells per well for 48 well or 96 well plates, respectively. Glial cells are seeded in parallel, before, or after attachment of iN cells in the same medium at densities between 60,000 and 120,000 or between 20,000 and 50,000 cells per well for 12-well or 48 well plates, respectively. Two days after seeding, half of the medium is replaced and AraC is added to final concentration of 2 μm in order to prevent overgrowth of glial cells. During the first week after seeding, half-medium changes are performed every other day. During the second week, half-medium changes are performed every 3 days, and afterwards, half-medium changes are performed twice a week and at least two days before recording of neuronal activity. Neural co-cultures on MEA plates can be maintained at 37° C. and 5% CO₂ for over 6 weeks.

In one aspect, the present invention provides a human neural cell co-culture that provides synchronous network bursts, the co-culture comprising: in vitro differentiated functional human neuronal cells; and glial cells, such as mouse, rat, or human glia cells. In general, the neural cell co-culture provided herein is characterized by being capable of forming synapses, and preferably generate, synchronous network bursts, which is observed about 2, 3, 4, or 5 weeks after the seeding of the co-culture. Synchronized network bursts if the first spikes of individual bursts are co-occurring within about 5, about 10, about 20, about 30, about 40 milliseconds; measured by at least about 25, 30, 35, 40, 45, 50, 55, 60, 65, 70, 75, 80, 85, 90, or 95% of active electrodes in any single well on a MEA plate.

In some embodiments, the present invention provides a human neural cell co-culture in which inhibitory/excitatory neuronal cell ratios can be modulated for enhancement of specific phenotypes of effects on network activity (FIG. 12A). Moreover, different time points of human neural co-culture maturation with different degrees or even absence of apparent neuronal network coordination may be chosen for analysis or agent exposure. This may enhance or unmask effects on network activity, e.g. inhibition of GABAergic signal transduction by bicuculine (FIG. 12B).

In some embodiments, the neural cells are seeded and maintained on plates with clear well bottoms, which can be used for image-based analyses (e.g. high-content imaging, see, for example, Opera Phenix High-Content Screening System from Perkin Elmer). The clear-bottom plates may be precoated with a suitable substrate, including without limitation laminin, PEI, PO, PDL, matrigel, etc.

Specific plating steps may comprise, for example the following steps: Before replating the iN cells and glial cells, the clear-bottom plates are pre-coated with matrigel or polyethylenimine (PEI) and laminin. Pure excitatory, pure inhibitory, or a mixture of excitatory and inhibitory iN cells in the presence of absence of glial cells are seeded at different densities depending on the type of assay, for example to reach a final ratio excitatory/inhibitory iN cells of 70%/30% for modeling physiological conditions. Neuronal cells are plated in neurobasal/B27 medium (Neurobasal/B27 medium: Neurobasal-A medium+B27+0.5×Glutamax+NT3 [10 ng/ml]+mouse laminin [200 ng/ml]) supplemented with 2 μg/ml doxycycline, 1% FBS, and 10 μm Rock inhibitor (Y27632). The total number of seeded iN cells may be in the range between 100,000 and 500,000 cells per well for 12-well plates, between 50,000 and 250,000 cells per well for 24-well plates, between 25,000 and 200,000 cells per well for 48-well plates, between 5,000 and 100,000 cells per well for 96-well plates, between 500 and 20,000 cells per well for a 384-well plate, respectively. Glial cells are seeded in parallel, before, or after attachment of iN cells in the same medium at densities between 25,000 and 250,000, between 12,000 and 125,000, between 6,000 and 100,000, between 1,200 and 50,000, ro between 100 and 10,000 cells per well for 12-well, 24-well, 48-well, 96-well, or 384-well plates, respectively. Two days after seeding, half of the medium is replaced and AraC is added to final concentration of 2 μm in order to prevent overgrowth of glial cells. During the first week after seeding, half-medium changes are performed every other day. During the second week, half-medium changes are performed every 3 days, and afterwards, half-medium changes are performed twice a week and at least two days before recording of neuronal activity. Neural co-cultures on clear-bottom plates can be maintained at 37° C. and 5% CO₂ for over 3 weeks.

In one aspect, the present invention provides functional and mature human neuronal and glial cell co-cultures capable of forming synapses, neuronal circuits, and neuronal network, the co-culture comprising: in vitro differentiated functional human neuronal cells; and glial cells, such as mouse, rat, or human glia cells.

### Analysis and Screening Methods

In some embodiments the provided assays are used to study the effects of biologically active candidate agents on neural development, function, cellular physiology, and cell-cell interactions. Candidate agents can include nucleic acids that produce altered gene functions or change expression levels of exogenous or endogenous transcripts, proteins, peptides, lipids, carbohydrates, as well as inorganic and organic chemicals. Of particular interest is to analyze chemicals that are exposed to humans and the environment, e.g. pesticides and material additives, as well as compounds intended for pharmaceutical use, e.g. antibodies and small molecule inhibitors, to identify neurotoxic effects and to assess efficacy, effectiveness, and off-target effects of new drugs, respectively.

In screening assays for biologically active agents, the effect of altering the environment of neurons in culture is tested, e.g. with a panel of cells and cellular environments. The effect of the altering of the environment is assessed by monitoring output parameters, including one or more of viability, propagation of action potentials and calcium release, outgrowth of dendrites, cell morphology, synaptic density, abundance and appearance of specific proteins, cell trafficking, cellular organelles, and the like. By being able to compare the effect on these parameters as to the degree of change in the absence of the compounds, the function of the compounds can be compared, the pathways affected identified and side effects predicted.

In screening assays for genetic agents, polynucleotides are added to one or more of the cells in a panel in order to alter the genetic composition of the cell. The output parameters are monitored to determine whether there is a change in phenotype affecting particular pathways. In this way, genetic sequences are identified that encode or affect expression of proteins in pathways of interest.

In some embodiments cells in the neuronal system comprise genetic changes typical of a condition of interest, for example to assess the effects of genotypes and agents on neuronal viability, function, and morphology, at a mature differentiation state using fully matured neuronal cells. This includes the study of genotypes that affect synaptic function in disorders such as schizophrenia and agents such as organophosphates, e.g. used in insecticides that impair specific synaptic transmissions. In other embodiments effects of genotypes and agents on neural development can be assessed using neural cultures with less differentiated neuronal cells. Thus, neurodevelopmental effects can be tested that either immediately affect function, maturation, and viability of developing cells, such as trimethyltin-derivates, or exhibit long-term effects emerging as phenotypes in mature neural cultures, such as organochlorine pesticides and their contribution to the etiology of autism.

In some embodiments the provided screening assays is combined with optogenetic methods to specifically and transiently activate or inhibit subpopulations of cells within the neural network. Activation of a specific subpopulation of cell types in the neural screening assay mix is achieved by exogenous expression of the light-sensitive cation channel channelrhodopsin-2 (ChR) or the like and optical excitation through application of light stimulation of the corresponding wavelength. Inhibition of a specific subpopulation of cell types in the neural screening assay mix is achieved by exogenous expression of the light-sensitive proton pump archaerhodopsin-3 (ArchT) or the like and optical perturbation through application of light stimulation of the corresponding wavelength.

In some embodiments cells in the neuronal/glial system comprise genetic changes typical of a condition of interest, for example to assess the effects of genotypes and agents on neuronal viability, function, morphology, and cell-cell interactions between neurons and glial cells. This includes the study of genotypes that affect neuronal health, e.g. by promoting protein aggregation in disorders such as Parkinson's, Huntington's or Alzheimer's disease or affecting established cell-cell interaction such as decreasing myelination of neurons as evident in diseases like multiple sclerosis. In other embodiments effects of genotypes can be assessed using neuronal cells and glial cells such as microglial, oligodendroglial, and astroglial cells. Thus, neurodegenerative effects can be tested that either immediately affect function, viability, and morphology or show delayed effects on neuronal physiology and function. Furthermore, agents can be tested for their capacity to ameliorate, prevent, or counteract such effects by acting on any of the incorporated cell types.

Conditions of neurodevelopmental and neuropsychiatric disorders and neural diseases that have strong genetic components or are directly caused by genetic or genomic alterations can be modeled with the provided assay. Genetic alterations include for example point mutations in genes such as NLGN1/3/4, NRXN1/4, SHANK2/3, GRIN2B, FMR1, or CHD8 that represent risk alleles for autism spectrum disorders, point mutations in or deletions of genes such as CACNA1, CACNB2, NLGN4X, LAMA2, DPYD, TRRAP, MMP16, NRXN1 or NIPAL3 that are associated with schizophrenia, mutations in the SCN1A gene that are related to seizure disorders, a triplet expansion in the HTT gene that cause to Huntington's disease (HD), monoallelic mutations in genes such as SNCA, LRRK2 and biallelic mutations in genes such as PINK1, DJ-1, or ATP13A2 that predispose to PD, single nucleotide polymorphisms (SNPs) in genes such as ApoE, APP, and PSEN1/2 that confer risks for developing Alzheimer's disease (AD) and other forms of dementia, as well as SNPs in genes such as CACNA1C, CACNB3, ODZ4, ANK3 that are strongly associated with bipolar disease (BP). Genomic alterations further include copy number variations (CNVs) such as duplications of 1q21.1, 7q11.23, 15q11.2, 22q11.2 or 16p11.2 that are associated with ASD, deletions of 15q13.3 or 16p11.2 that are associated with ASD, duplications of 16p13.11 or 16q11.2 that are associated with schizophrenia, and deletions of 15q11.2 or 22q11.21 that are associated with schizophrenia. Neurological disorders and neural diseases can also be driven by epigenetic alteration that can, for example, be caused by a trinucleotide expansion in the first exon and subsequent chromatin silencing of the FMR1 gene, which constitutes the underlying pathomechanism of fragile X syndrome (FXS). For these purposes, disease-associated or disease-causing genotypes can be generated in healthy iPS cells through targeted genetic manipulation or iPS cells can be derived from individual patients that carry a recurrent disease-related genotype and are diagnosed with the corresponding disease. Moreover, neural diseases with less defined or without genetic components can be modeled by selective perturbation or excitation of specific cell populations within the neural network, e.g. inhibition of the inhibitory neurons in a mixed neuronal network to mimic seizures.

Neuronal activity of human neural co-cultures can be assessed by combining the neural co-culture with a monitoring device. In some embodiments the monitoring device measures extracellular currents and local field potentials, e.g. using MEA systems. In alternative embodiments, other methods that measure synchronized network activity, e.g. Ca++ sensitive dyes, patch clamping, or any other method of measuring current and local field potentials.

The monitoring device may be based on electrical detection of extracellular currents and field potentials using electrodes incorporated in the bottom of cell culture plates and subsequent amplification and processing of detected signals such as multielectrode arrays (MEAs), e.g. as shown in FIG. 1B, elements 1, 4 and 5. Alternatively, calcium (Ca²⁺) imaging can be applied to measure changes in the intracellular Ca²⁺ concentration of neuronal cells indicative of neuronal activity. Here, both chemical indicators and genetically encoded indicators that change their cellular distribution or optical characteristics upon Ca²⁺ binding can be used. Furthermore, voltage-sensitive dyes that change their spectral properties in response to voltage changes and therefore indicate changes in membrane potentials of neuronal cells can be applied to measure neuronal activity. Changes in light extinction, absorption, or emission can then be captured by CCD cameras and microscope devices. The property of synchronous firing can tested using the same monitoring devices by the electrophysiological recording of the whole culture using MEAs or imaging of neuronal activity using Ca²⁺ indicators or voltage-sensitive dyes.

For detection of action potentials in neural cultures, signals can be detected as spikes when exceeding a present voltage increase, e.g. 2×, 3×, 4×, 5×, 6× or more the standard deviation of voltages measured by each electrode. A set of sequential spikes may be defined as a burst if at least about 3, about 4, about 5 or more spikes are detected by one electrode within a defined period of time, e.g. from around about 10-500 milliseconds, around about 50 to about 250 millisecond, or around about 100 milliseconds. Bursts detected across multiple electrodes per well can be defined as synchronized network bursts if the first spikes of individual bursts are co-occurring within about 5, about 10, about 20, about 30, about 40 milliseconds; measured by at least 25, 35, 45, 50, 65, 75% of active electrodes.

In some embodiments the data is obtained from an array of microelectrodes. For example, the Maestro MEA platform from Axion BioSystems can be used to maintain neural co-cultures in a medium-to-high throughput format (96-well plates available, 384-wells announced) for versatile screening applications. Here, neuronal activity of neural co-cultures grown on MEA plates can be measured by a total of 768 electrodes distributed over 12, 48, or 96 wells generating up to 12,500 data points per second. Recordings are performed in a temperature-controlled environment (37° C.) on the Maestro base unit and input signals are being processed by the Middleman unit. For detection of action potentials in neural cultures, a standard configuration provided by the operating software AxIS (Neural Spike mode) can be applied using a sampling frequency of 12.5 kHz, a voltage scale of 5.5×10-8 V per sample, and a bandpass filter from 200 Hz to 3 kHz. Here, signals are detected as spikes when exceeding 6× the standard deviation of voltages measured by each electrode. For most assay applications, a recording period of about 10 minutes can be carried out generating an output file including spike information of the format .spk. Subsequent analysis and compilation of generated output files can be performed using statistical programming language packages (e.g. R Bioconductor) or graphical use interface software tools such as NeuroMetricTool (provided by Axion BioSystems). For most purposes and general characterization of neuronal activity, a set of sequential spikes can be defined as a burst if at least 5 spikes are detected by one electrode within 100 milliseconds. Bursts detected across multiple electrodes per well can be defined as synchronized network bursts if the first spikes of individual bursts are co-occurring within 20 milliseconds measured by at least 50% of active electrodes (FIG. 2B). For characterization of phenotypes of neuronal activity various readout parameters as described herein can be applied.

Neuronal morphology, cellular trafficking, cellular organelles, and cell-cell interaction, of and within human neuronal/glial co-cultures, as well as abundance, distribution, aggregation, and interaction of specific proteins can be assessed by combining the provided co-culture with an optical monitoring device. In some embodiments the monitoring device measures abundance, intensity, and localization of light signals attached to membranes, or proteins or measures light emission of reporter systems, e.g. using high-content imaging systems with immunofluorescence staining and fluorescent resonance energy transfer (FRET). In alternative embodiments, other methods that measure enzyme activity, substrate concentration, and substrate conversion in the co-culture or the supernatant, e.g. colorimetric, fluorescence, or luminescence readouts, can be applied

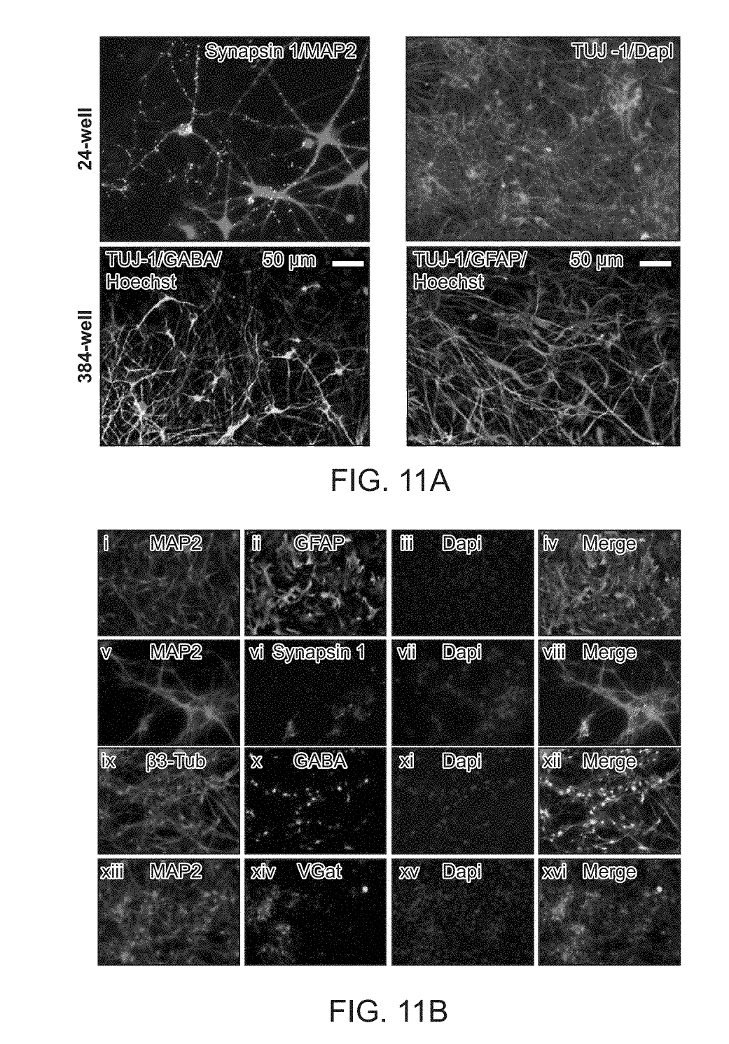

The monitoring device may be based on confocal or wide-filed image acquisition of fluorescently labelled proteins in the neuronal/glial co-culture (FIG. 11). Labeling of proteins, e.g. using specific antibodies conjugated to fluorescence probes or fluorescence protein fusion proteins, can be used to measure the overall abundance and spatial distribution of a specific single proteins or a protein complex as well as observed changes upon genetic or chemical treatment. Furthermore, protein labeling may be used to determine cellular morphology, e.g. structural proteins like MAP2 (FIGS. 11Bi, v, ix, xiii), synapse formation, e.g. staining synaptic proteins like synapsin1 (FIGS. 11A and Bvi), HOMER, bassoon, synaptophysin and PSD95, cellular organelles, e.g. autophagosome staining against LC3, cellular trafficking, e.g. using fluorescence recovery after photobleaching (FRAP) or live cell imaging of tagged proteins, and cell-cell interaction, e.g. co-localization of different labeled proteins. The overall abundance, intensity, and local distribution of light emission of different wave lengths from different labels can then be captured by CCD cameras and microscope devices. Acquired images can then be subjected to manual, semi-automated, or fully automated image analyses including quantification of pixel values, localization of pixel values, structural pattern recognition, cellular and subcellular regionalization, and time-dependent changes. In other embodiments, the monitoring device may be a fluorescence, luminescence, or photometric plate reader for measuring light signal intensities or colorimetric changes integrated over a whole well.

### Data and Screening Analysis

Comprehensive measurements of neuronal activity using electrical or optical recordings of the parameters described herein may include spontaneous activity and activity in response to targeted electrical or optical stimulation of all neuronal cells or a subpopulation of neuronal cells within the network. Furthermore, spontaneous or induced neuronal activity can be measured in the self-assembled functional environment and circuitry of the neural culture or under conditions of selective perturbation or excitation of specific subpopulations of neuronal cells as discussed above.

In the provided assays, comprehensive measurements of neuronal activity acquired by electrical recordings through MEAs or visual recordings of Ca release and voltage dependent probes can be conducted at different time points along neuronal maturation and usually include a baseline measurement directly before contacting the neural culture with the agents of interest and a subsequent measurement under agent exposure. Moreover, long-term effects of agents on neural maturation and development can be assessed by contacting the immature neural culture at an early time point with agents of interest and acquiring measurements of the same cultures after further maturation at a later time point compared to control cultures without prior agent exposure.

In some embodiments, standard recordings of neuronal activity of mature neural cultures are conducted after about 2 weeks, after about 3 weeks, after about 4 weeks of co-culture (i.e. after mixing the different cell components of the culture), at a time where synchronized firing of neuronal networks is robustly observed. The recordings of neuronal activity where a test agent is present can be conducted with different time frames including short recordings of e.g. 15 minutes to measure acute effects or long recordings of e.g. 60 minutes to identify delayed effects. Measurements for every assay condition may be conducted in parallel for 5-8 replicate cultures. Recordings of neuronal activity may encompass the measurement of additive, synergistic or opposing effects of agents that are successively applied to the cultures, therefore the duration recording periods can be adjusted according to the specific requirements of the assay. In some embodiments the measurement of neuronal activity is performed for a predetermined concentration of an agent of interest, whereas in other embodiments measurements of neuronal activity can be applied for a range of concentrations of an agent of interest.

In some embodiments the provided assays are used to assess general viability of the neural culture or single components including astroglial cells, oligodendroglial cells, and subtypes of neuronal cells where the single components can be cultured in defined mixes or as homogenous cell populations. Here, viability can be measured by quantitation of intracellular ATP, extracellular release of adenylate kinase, activation of proapoptotic proteins, e.g. caspase 3 and 7, staining with 7-Aminoactinomycin and Annexin V, staining with calcein AM and EthD-1 and the like. Assessment of viability is conducted as endpoint measurements at time points that can vary based on the compound of interest. Measurements can be conducted using luminescence readers, FACS analysis, immunoblotting, or fluorescence microscopy imaging.

In some embodiments the provided assays are used to assess maturation of the neural culture or single components including astroglial cells, oligodendroglial cells, and subtypes of neuronal cells where the single components can be cultured in defined mixes or as homogenous cell populations. Maturation of astroglial cells can be measured by expression of marker proteins including GFAP, S100β, and CD44 alone or in combination using FACS analysis, immunoblotting, or fluorescence microscopy imaging. Maturation of oligodendroglial cells can be measured by morphology and expression of marker proteins such as O4, Olig2 and MBP alone or in combination using FACS analysis, immunoblotting, or fluorescence microscopy imaging. Maturation of neuronal cells can be measured based on morphology by optically assessing parameters such as dendritic arborization, axon elongation, total area of neuronal cell bodies, number of primary processes per neuron, total length of processes per neuron, number of branching points per primary process as well as density and size of synaptic puncta stained by synaptic markers such as synapsin-1, synaptophysin, bassoon, PSD95, and homer. Moreover, general neuronal maturation and differentiation can be assessed by measuring expression of marker proteins such as MAP2, TUJ-1, NeuN, Tau, PSA-NCAM, and synapsin-1 alone or in combination using FACS analysis, immunoblotting, or fluorescence microscopy imaging. Maturation and differentiation of neuronal subtypes can further be tested by measuring expression of specific proteins. For excitatory neuronal cells this includes staining for e.g. vGlut1/2, GRIA1/2/3/4, GRIN1, GRIN2A/B, and ChAT. For inhibitory neuronal cells this includes staining for e.g. GABRA2, GABRB1, vGAT, and GAD67. For dopaminergic neuronal cells this includes staining for e.g. TH, Nurr1, LMX1B, and GIRK2.

Agents are screened for biological activity by adding the agent to at least one and usually a plurality of co-culture wells to form a panel of assay combinations (where an assay combination may be defined as the specific cell combination, media, and agent present in an assay), usually in conjunction with assay combinations lacking the agent. The change in parameter readout in response to the agent is measured, desirably normalized, and the resulting dataset may then be evaluated by comparison to reference data. The reference data may include basal readouts in the presence and absence of the factors, data obtained with other agents, which may or may not include known inhibitors of known pathways, etc. Agents of interest for analysis include any biologically active molecule with the capability of modulating, directly or indirectly, the phenotype of interest of a cell of interest.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method. Genetic agents may be added to pluripotent cells prior to neuronal induction and glial differentiation in order to produced cell populations or monoclonal cell sublines with specifically altered genome content.

In the provided neuronal screening system, parameters of neuronal activity may also be measured in neuronal cells derived from different human individuals to study phenotypic differences related to different genetic backgrounds. Moreover, parameters of neuronal activity may be measured in cells derived from healthy individuals and cells derived from patients suffering from a disease of interest carrying one or multiple genetic factors for the disease. Agents may then be added to both cells from healthy individuals and affected patients to assess relative changes in neuronal activity. Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying parameters. Parameters of baseline neuronal activity and changes in neuronal activity after treatment with agents or differences in neuronal activity between cells from individuals with different genetic backgrounds can be measured by electrical methods detecting extracellular currents and local field potentials, such as multielectrode arrays (MEAs). Therefore, the human neural co-culture is typically grown on specialized culture plates with microelectrodes integrated in the bottom of the well. Electrical signals of single or groups of cells in the proximity are then detected, amplified and processed to identify action potentials (spikes) and quantify parameters informative of neuronal activity.

Parameters of neuronal activity can also be quantified by optical methods detecting changes in intercellular calcium (Ca²⁺) concentrations (calcium imaging) or changes in the cell membrane potential of neuronal cells (voltage-sensitive dyes). For measuring neuronal activity through changes in the Ca²⁺ concentration chemical indicators that change their cellular distribution or optical characteristics upon Ca²⁺ binding, e.g. fura-2 or indo-1, as well as genetically encoded indicators that change fluorescence properties upon Ca²⁺ binding, e.g. GCaMP, can be used. Typically, chemical indicators are added to the neuronal screening system shortly before measuring neuronal activity and genetically encoded indicators added to the cells prior to neuronal induction. For measuring neuronal activity through changes in the membrane potential voltage-sensitive dyes that change their spectral properties in response to voltage changes can be applied, e.g. di-4-ANEPPS or RH237. Voltage-sensitive dyes are typically added to the neuronal screening system shortly before the measurement takes place. For optical detection of neuronal activity, depending on type of optical probe, CCD cameras can be used in conjunction with microscope devices, including fluorescence microscopes, to record changes in light extinction, absorption, or emission. Confocal and two-photon microscopes can be used to increase spatial resolution. Optical signals can then be subjected to computational processing to delineate action potentials (spikes) and quantify neuronal activity.

For measuring the amount, localization, or molecular interaction of a parameter molecule that is present, e.g. a protein, mRNA, glycan, etc., a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. For measuring total and relative amounts of mRNA, common methods include next generation sequencing and microarray hybridization.

The use of high affinity antibody binding and/or structural linkage during labeling provides dramatically reduced nonspecific backgrounds, leading to clean signals that are easily detected. Such extremely high levels of specificity enable the simultaneous use of several different fluorescent labels, where each preferably emits at a unique color. Fluorescence technologies have matured to the point where an abundance of useful dyes are now commercially available. These are available from many sources, including Sigma Chemical Company (St. Louis Mo.) and Molecular Probes (Handbook of Fluorescent Probes and Research Chemicals, Seventh Edition, Molecular Probes, Eugene Oreg.). Other fluorescent sensors have been designed to report on biological activities or environmental changes, e.g. pH, calcium concentration, electrical potential, proximity to other probes, etc. Methods of interest include calcium flux, nucleotide incorporation, quantitative PAGE (proteomics), etc.

Multiple fluorescent labels can be used on the same sample and individually detected quantitatively, permitting measurement of multiple cellular responses simultaneously. Many quantitative techniques have been developed to harness the unique properties of fluorescence including: direct fluorescence measurements, fluorescence resonance energy transfer (FRET), fluorescence polarization or anisotropy (FP), time resolved fluorescence (TRF), fluorescence lifetime measurements (FLM), fluorescence correlation spectroscopy (FCS), and fluorescence photobleaching recovery (FPR) (Handbook of Fluorescent Probes and Research Chemicals, Seventh Edition, Molecular Probes, Eugene Oreg.).

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

The results of an assay can be entered into a data processor to provide a dataset. Algorithms are used for the comparison and analysis of data obtained under different conditions. The effect of factors and agents is read out by determining changes in multiple parameters. The data will include the results from assay combinations with the agent(s), and may also include one or more of the control state, the simulated state, and the results from other assay combinations using other agents or performed under other conditions. For rapid and easy comparisons, the results may be presented visually in a graph, and can include numbers, graphs, color representations, etc.

The dataset is prepared from values obtained by measuring parameters in the presence and absence of different cells, e.g. genetically modified cells, cells cultured in the presence of specific factors or agents that affect neuronal function, as well as comparing the presence of the agent of interest and at least one other state, usually the control state, which may include the state without agent or with a different agent. The parameters include functional states such as synapse formation and Ca⁺⁺ release in response to stimulation, whose levels vary in the presence of the factors. Desirably, the results are normalized against a standard, usually a “control value or state,” to provide a normalized data set. Values obtained from test conditions can be normalized by subtracting the unstimulated control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a dataset may comprise normalized data from one, two or multiple cell types and assay conditions.

The dataset can comprise values of the levels of sets of parameters obtained under different assay combinations. Compilations are developed that provide the values for a sufficient number of alternative assay combinations to allow comparison of values.

A database can be compiled from sets of experiments, for example, a database can contain data obtained from a panel of assay combinations, with multiple different environmental changes, where each change can be a series of related compounds, or compounds representing different classes of molecules.

Mathematical systems can be used to compare datasets, and to provide quantitative measures of similarities and differences between them. For example, the datasets can be analyzed by pattern recognition algorithms or clustering methods (e.g. hierarchical or k-means clustering, etc.) that use statistical analysis (correlation coefficients, etc.) to quantify relatedness. These methods can be modified (by weighting, employing classification strategies, etc.) to optimize the ability of a dataset to discriminate different functional effects. For example, individual parameters can be given more or less weight when analyzing the dataset, in order to enhance the discriminatory ability of the analysis. The effect of altering the weights assigned each parameter is assessed, and an iterative process is used to optimize pathway or cellular function discrimination.

### Candidate Agents

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants and fungi. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Genetic Agents

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; encode a dominant negative mutation, or dominant or constitutively active mutations of native sequences; altered regulatory sequences, etc.

In addition to sequences derived from the host cell species, other sequences of interest include, for example, genetic sequences of pathogens, for example coding regions of viral, bacterial and protozoan genes, particularly where the genes affect the function of human or other host cells. Sequences from other species may also be introduced, where there may or may not be a corresponding homologous sequence.

Genetic agents also include the introduction of guide sequences and enzymes of the CRISPR/Cas9 technology. CRISPR/Cas9 can directly cleave specific genomic sequences leading to incorrect double strand break repair and thus frame shift mutations disrupting endogenous genes or regulatory regions. In addition, Cas9-derivatives can specifically bind to genomic loci and with the use of attached/fused proteins or functional protein domains (e.g. histone demethylase LSD1 or VP64 transactivator domain) either modify local chromatin or directly regulate gene expression.

A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746.

cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

In one embodiment, the genetic agent is an antisense sequence that acts to reduce expression of the complementary sequence. Antisense nucleic acids are designed to specifically bind to RNA, resulting in the formation of RNA-DNA or RNA-RNA hybrids, with an arrest of DNA replication, reverse transcription or messenger RNA translation. Antisense molecules inhibit gene expression through various mechanisms, e.g. by reducing the amount of mRNA available for translation, through activation of RNAse H, or steric hindrance. Antisense nucleic acids based on a selected nucleic acid sequence can interfere with expression of the corresponding gene. Antisense nucleic acids can be generated within the cell by transcription from antisense constructs that contain the antisense strand as the transcribed strand.

The anti-sense reagent can also be antisense oligonucleotides (ODN), particularly synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene. A combination of sequences may also be used, where several regions of the mRNA sequence are selected for antisense complementation.

Antisense oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH.sub.2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs. The .alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity.

As an alternative method, dominant negative mutations are readily generated for corresponding proteins. These may act by several different mechanisms, including mutations in a substrate-binding domain; mutations in a catalytic domain; mutations in a protein binding domain (e.g. multimer forming, effector, or activating protein binding domains); mutations in cellular localization domain, etc. See Rodriguez-Frade et al. (1999) P.N.A.S. 96:3628-3633; suggesting that a specific mutation in the DRY sequence of chemokine receptors can produce a dominant negative G protein linked receptor; and Mochly-Rosen (1995) Science 268:247.

Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

### Data Analysis

The collected data from measurements of viability, neural maturation, and the different modes of neuronal activity recordings acquired from the provided neuronal screening system for each condition can be integrated in a comprehensive delineation of neurobiological characteristics to generate a specific phenotype profile for a certain agent or genotype. Such a profile can for example combine alterations in cellular structure and morphology in specific components of the neural culture (e.g. reduced synapse density for inhibitory neuronal cells compared to untreated cells) and functional changes in neuronal activity (e.g. lower enhancement of synchronous burst activity upon specific perturbation of the inhibitory neuronal cell component of the network compared to untreated cells) while including parameters of activity, viability and maturation that stay unchanged under treatment and control conditions.

Specific phenotype profiles for tested agents and genotypes generated by the provided assays and the methods described above can be stored in a database to archive biological effects on neural function in a comparable biologically meaningful description. Stored phenotype profiles can then be matched according to inverse behavior to identify agents that counteract effects of know neurotoxic substances or the phenotypes observed for neural diseases and psychological disorders modeled by targeted genetic manipulation or patient derived cells, as discussed above. This allows for in silico analyses of potential treatments and modes of actions as well as for preselection of compound classes as candidates for drug development.

The comparison of a dataset obtained from a test compound, and a reference dataset(s) is accomplished by the use of suitable deduction protocols, AI systems, statistical comparisons, etc. Preferably, the dataset is compared with a database of reference data. Similarity to reference data involving known pathway stimuli or inhibitors can provide an initial indication of the cellular pathways targeted or altered by the test stimulus or agent.

A reference database can be compiled. These databases may include reference data from panels that include known agents or combinations of agents that target specific pathways, as well as references from the analysis of cells treated under environmental conditions in which single or multiple environmental conditions or parameters are removed or specifically altered. Reference data may also be generated from panels containing cells with genetic constructs that selectively target or modulate specific cellular pathways. In this way, a database is developed that can reveal the contributions of individual pathways to a complex response.

The effectiveness of pattern search algorithms in classification can involve the optimization of the number of parameters and assay combinations. The disclosed techniques for selection of parameters provide for computational requirements resulting in physiologically relevant outputs. Moreover, these techniques for pre-filtering data sets (or potential data sets) using cell activity and disease-relevant biological information improve the likelihood that the outputs returned from database searches will be relevant to predicting agent mechanisms and in vivo agent effects.

For the development of an expert system for selection and classification of biologically active drug compounds or other interventions, the following procedures are employed. For every reference and test pattern, typically a data matrix is generated, where each point of the data matrix corresponds to a readout from a parameter, where data for each parameter may come from replicate determinations, e.g. multiple individual cells of the same type. As previously described, a data point may be quantitative, semi-quantitative, or qualitative, depending on the nature of the parameter.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e. data matrices) obtained from multiple repeated experiments. Classification rules are selected as correctly identifying repeated reference patterns and successfully distinguishing distinct reference patterns. Classification rule-learning algorithms may include decision tree methods, statistical methods, naive Bayesian algorithms, and the like.

A knowledge database will be of sufficient complexity to permit novel test data to be effectively identified and classified. Several approaches for generating a sufficiently encompassing set of classification patterns, and sufficiently powerful mathematical/statistical methods for discriminating between them can accomplish this.

The data from cells treated with specific drugs known to interact with particular targets or pathways provide a more detailed set of classification readouts. Data generated from cells that are genetically modified using over-expression techniques and anti-sense techniques, permit testing the influence of individual genes on the phenotype.

A preferred knowledge database contains reference data from optimized panels of cells, environments and parameters. For complex environments, data reflecting small variations in the environment may also be included in the knowledge database, e.g. environments where one or more factors or cell types of interest are excluded or included or quantitatively altered in, for example, concentration or time of exposure, etc.

The advantage of this invention over existing systems for neuronal screening is the ability to analyze effects of genotypes or agents on biologically relevant higher order neuronal functions of pure human neural cultures in a medium-to-high throughput manner. This invention combines different methods to produce neural cells by either using step-wise differentiation protocols to generate glial cells or directly inducing neuronal cell identities from human pluripotent stem cells through forced expression of neurogenic transcription factors and thereby dramatically accelerating neuronal maturation. The approach of combining single neural components from separate homogenous cell populations in different compositions and ratios creates a unique flexibility in setting up neural assays tailored to address specific questions. The resulting mixed defined neural cultures are marked by an unparalleled maturity and functionality of the neuronal component, as evidenced by the unique feature to develop spontaneous synchronized network activity in human neurons after only 3 weeks of culture. The highly functional neural cultures are then combined with a medium-to-high throughput analysis platform (MEAs) to measure neuronal activity in real time under experimental conditions. The combinatorial approach outlined by this invention provides a powerful tool to study basic biological functions, assess neurotoxic effects of pharmaceutical compounds and chemical substances, and support drug development against neural diseases and neurological disorders.

### Kits

For convenience, the systems of the subject invention may be provided in kits. The kits could include the appropriate additives for providing the simulation, optionally include the cells to be used, which may be frozen, refrigerated or treated in some other manner to maintain viability, reagents for maintaining the neural co-culture system, reagents for measuring the parameters, and software for preparing the data analysis.

### EXPERIMENTAL

### Example 1

### Development of Spontaneous Synchronized Network Activity in Neural Co-Cultures Consisting of Primary Glial Cells and Glutamatergic Excitatory iN Cells Measured on Multielectrode Arrays (MEAs)

Induced excitatory neurons were seeded at day 4 after induction by transcriptional activation of the neurogenic transcription factor NGN2, on 12-well MEA plates (Axion BioSystems) coated with matrigel. A total of 600,000 iN cells were plated per well. Primary glial cells were obtained by dissociating brains of mouse pups at postnatal day 3 with hippocampal and cerebellar structures being removed in advance. Dissociated brains were pre-cultured and passaged twice to remove primary neurons. Glial cells were then seeded directly on the plated iN cells at a density of 120,000 cells per well.

Neuronal activity was recorded using the Axion BioSystems MEA system set to detect neural spikes applying a bandpass filter from 200 Hz to 3 kHz (Neural Spikes mode). Recordings of spontaneous neuronal activity were performed at 1, 2, 3, and 4 weeks after plating for a period of 10 minutes each.

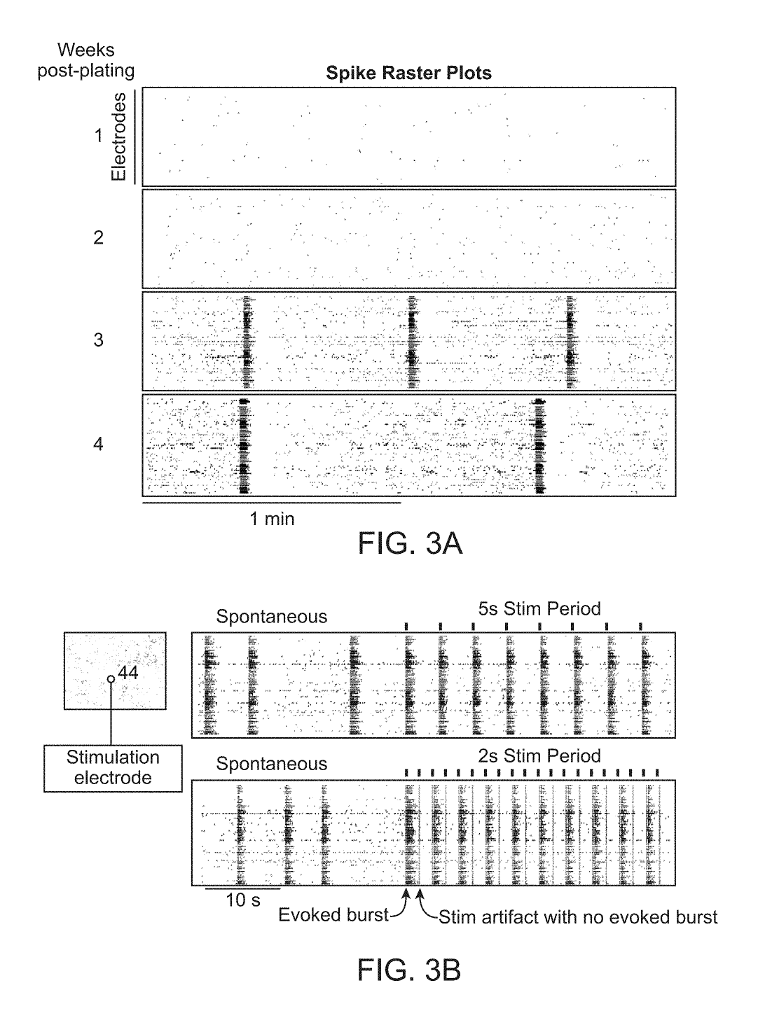

At week 1 after plating, rare spontaneous single spikes were detected by around 30% of the 64 electrodes across the well (FIG. 3A, 1^{st} panel). At week 2 after plating, spontaneous single spikes were detected by around 60% of the 64 electrodes across the well measured with an increased frequency (FIG. 3A, 2^{nd} panel). At week 3 after plating, spontaneous single spikes were detected by over 90% of the 64 electrodes across the well with strongly increased frequencies compared to week 1 and 2. Moreover, spontaneous synchronized network activity was observed across all active electrodes was (FIG. 3A, 3^{rd} panel). At week 4 after plating, spontaneous synchronized network activity was still detectable across all active electrodes (>90%) and the frequency of spontaneous single spikes in-between network burst was decrease compared to week 3 (FIG. 3A, 4^{th} panel).

Subsequent to recordings of spontaneous synchronized network activity at week 4, a second recording was performed to test the firing behavior of the neuronal network in response to electrical stimulation. For this purpose, a series of stimuli with 5 second intervals was applied to one of the 64 electrodes. As shown in FIG. 3B (upper panel) the neuronal network exhibited synchronous bursts in response to every single stimulus. The depolarization of neuronal membranes as a result of activation causes a rapid inactivation of Na+ channels, thereby terminating the action potential. Repeated activation of a neuron, e.g. during bursts of action potentials, leads to a slow inactivation of Na+channels, a natural mechanism to prevent hyperexcitability, which generates a quiescent period between bursts. In order to test this property in the neurons of the functional network, we applied a series of stimuli with 2 second intervals to one of the 64 electrodes.

As depicted in FIG. 3B (lower panel), the network exhibited synchronous bursts only in response to every other stimulus, suggesting an excitability of the prevailing neuronal network that resembles physiological conditions. These data indicated a fast maturation of the neuronal component of the neural co-culture with functional neuronal networks being formed at week 3 after plating (˜3½ weeks after induction). The occurrence of spontaneous synchronized network activity and the response of the neuronal network to applied electrical stimulation further implicates the presence of functional synapses, spontaneous action potentials, and demonstrates the ability of the cells to fire action potentials upon excitation. Moreover, the decrease in inter-network burst spike frequency suggested a continuing maturation of the neuronal network past week 3.

In a second experiment, the development of spontaneous synchronized network activity was further characterized and changes in basic parameters over time were quantified using MEAs. For this purpose, a total of 400,000 iN cells were plated per well of a 12-well MEA plate together with 100,000 primary glial cells following the aforementioned procedures. Accordingly, neuronal activity was recorded using the Axion BioSystems MEA system at 1, 2, 3, and 4 weeks after plating for a period of 10 minutes each.

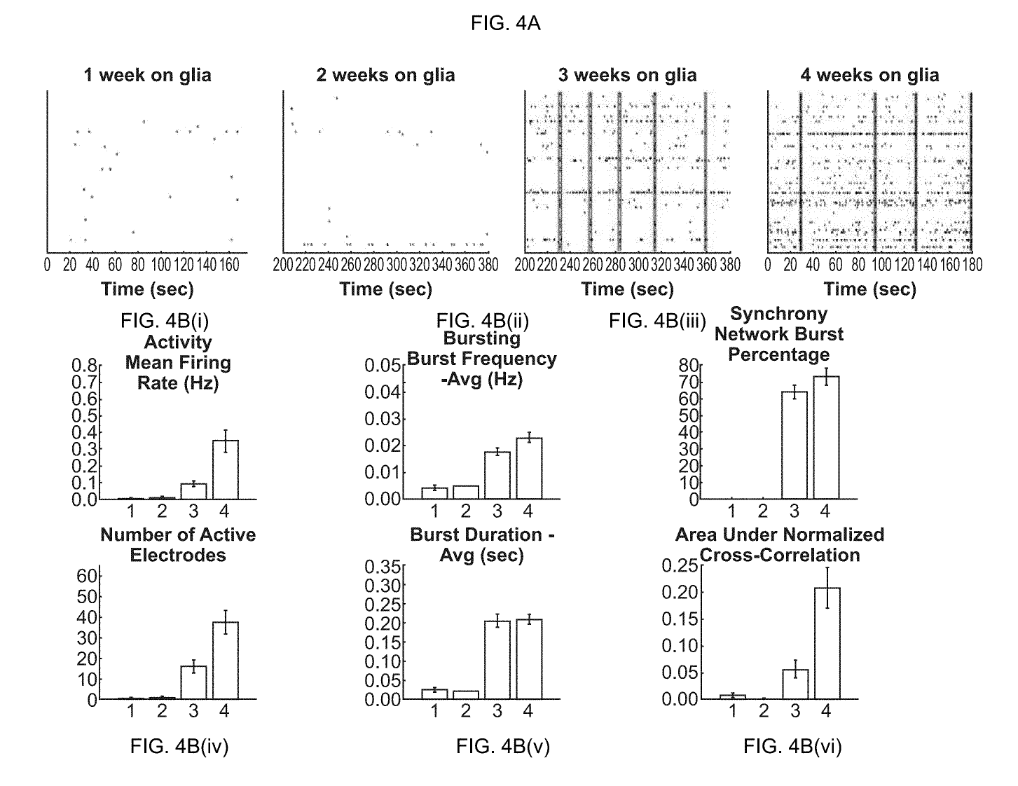

The observations of increasing spontaneous single spike frequencies from week 1 until week 4 and the formation of spontaneous synchronized network activity starting at week 3 corresponded to the previous experiment using 600,000 excitatory neurons co-cultures with 120,000 glial cells (FIG. 4A). The basic neuronal activity measured by the mean firing rate of spontaneously occurring spikes and the number of active electrodes (at least 5 spikes detected per minute) detecting active neurons (or clusters of neurons) steadily increased over time and reached around 0.35 Hz and 60%, respectively, after 4 weeks in culture (FIG. 4B, i and iv).

The occurrence of bursts of action potentials remained low during the first two weeks in culture and rapidly increased together with the formation of spontaneous synchronized network activity, as measured by burst frequency (Hz) and average burst duration (seconds) (FIG. 4B, ii and v). A set of sequential spikes was regarded as a burst if at least 5 spikes were detected by the same electrode within 100 milliseconds. In concordance with this, spontaneous synchronized network activity was detected starting weeks 3 measured by the percentage of total bursts occurring within a network burst. In order to be considered synchronized network bursts, co-occurring bursts needed to be detected with a time window of 20 milliseconds (first spike of each burst) in at least 50% of active electrodes (compare FIG. 2B). At week 3 and 4, around 62% and 74% of the bursts were part of spontaneous synchronized network activity (FIG. 4B, iii).

### Example 2

### Development of Spontaneous Synchronized Network Activity in Neural Co-Cultures Consisting of Primary Glial Cells and a Mixture of Glutamatergic Excitatory iN Cells and GABAergic Inhibitory iN Cells Measured on MEAs

Induced excitatory neurons were seeded at day 4 and induced inhibitory neurons were seeded at day 6 after induction. A total of 200 000 excitatory iN cells and 200 000 inhibitory iN cells were plated simultaneously per well on 12-well MEA plates (Axion BioSystems) coated with matrigel. Primary glial cells were obtained as described in Example 1, and seeded directly on the plated iN cells at a density of 100 000 cells per well.

Inhibitory iN cells showed a significantly higher apoptosis rate than excitatory iN cells after plating which resulted in an approximate ratio of 70%/30% (excitatory/inhibitory) after 2 weeks in culture, thereby reflecting the actual ratio present in most regions of the human brain. Neuronal activity was recorded using the Axion BioSystems MEA system set to detect neural spikes applying a bandpass filter from 200 Hz-3 kHz (Neural Spikes mode).

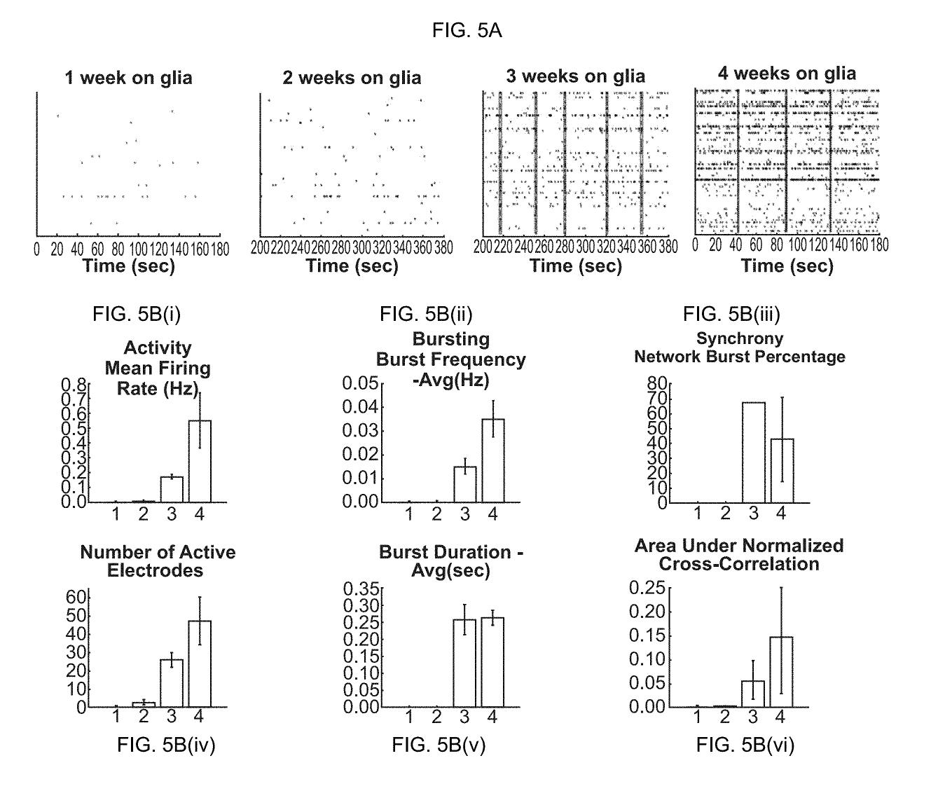

Recordings of spontaneous neuronal activity were performed at 1, 2, 3, and 4 weeks after plating for a period of 10 minutes each. The threshold values for defining active electrodes, bursts and spontaneous synchronized network activity were the same as described under Example 1. In accordance with the pure excitatory co-cultures, the basic neuronal activity measured by the mean firing rate of spontaneously occurring spikes and the number of active electrodes (at least 5 spikes detected per minute)) steadily increased over time and reached around 0.55 Hz and 50%, respectively, after 4 weeks in culture (FIGS. 5A, and 5B, i and iv). Bursts of action potentials were only detected at week 3 and 4 and increased in frequency from around 0.017 to 0.035 Hz (FIG. 5B, ii). In contrast, the burst duration remain constant at around 0.25 seconds (FIG. 5B, v). Spontaneous synchronized network activity was detected starting weeks 3 with around 70% of bursts occurring during synchronous network bursts and decreased until weeks 4 with 40% of bursts occurring as synchronous network activity (FIG. 5B, iii). These data demonstrate that mixed excitatory/inhibitory neural co-cultures are capable of forming spontaneous synchronized network activity.

### Example 3

### Effects of Chemical Compounds on Neuronal Network Activity in Neural Co-Cultures Consisting of Primary Glial Cells and Glutamatergic Excitatory iN Cells Measured on MEAs

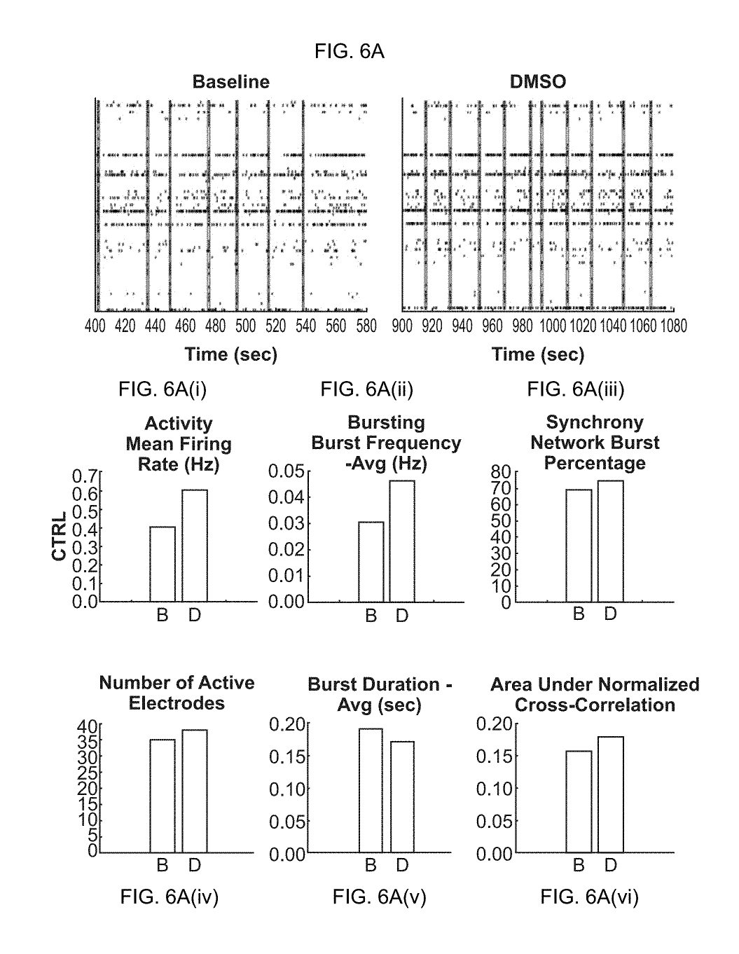

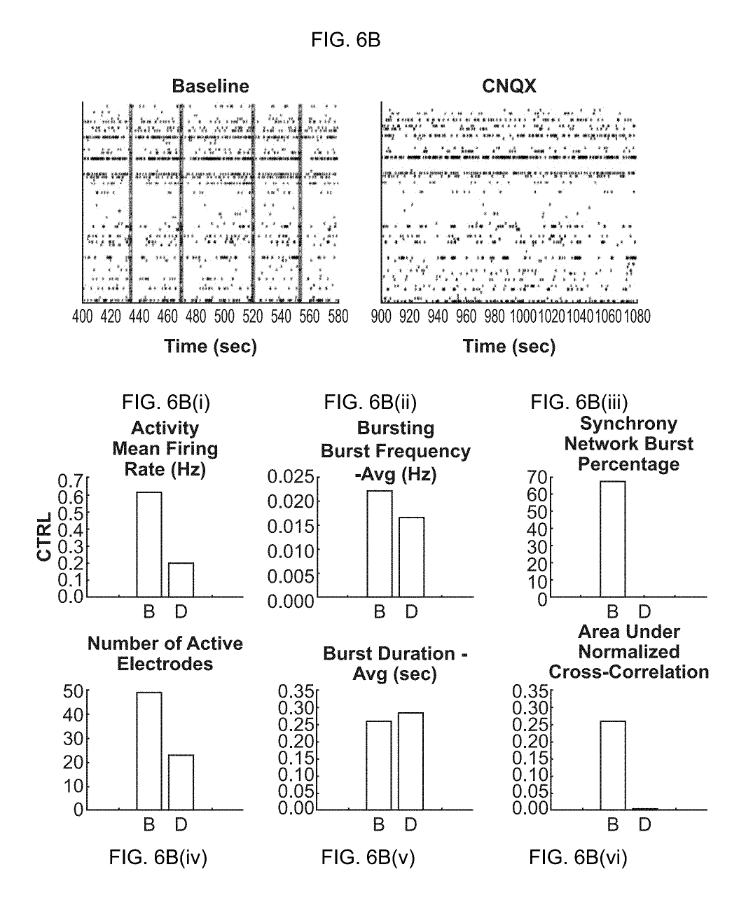

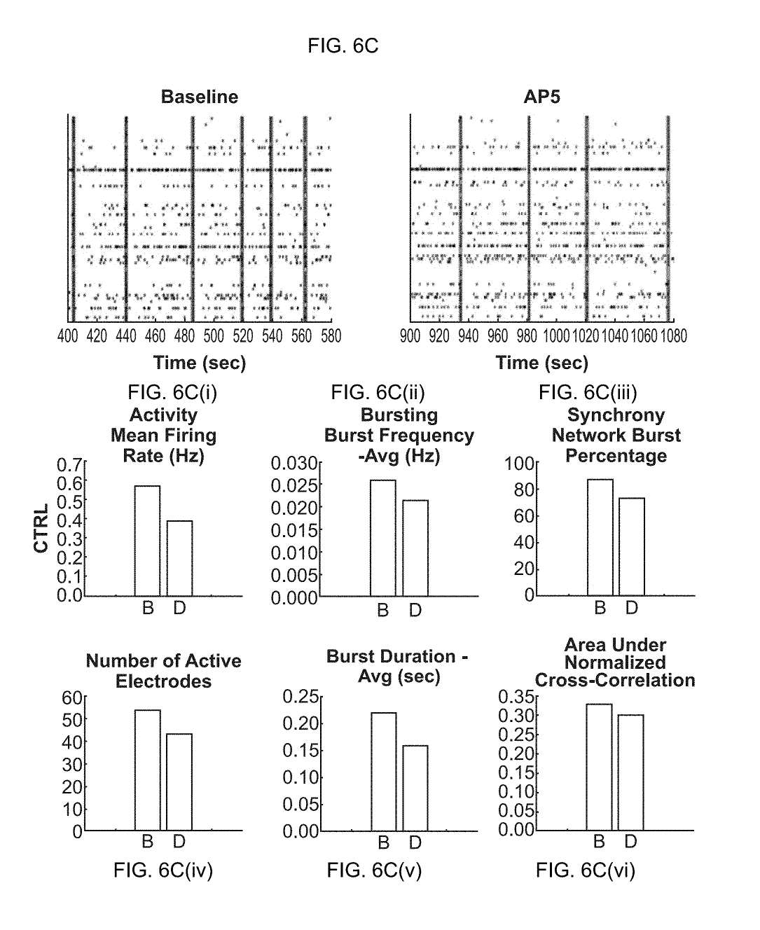

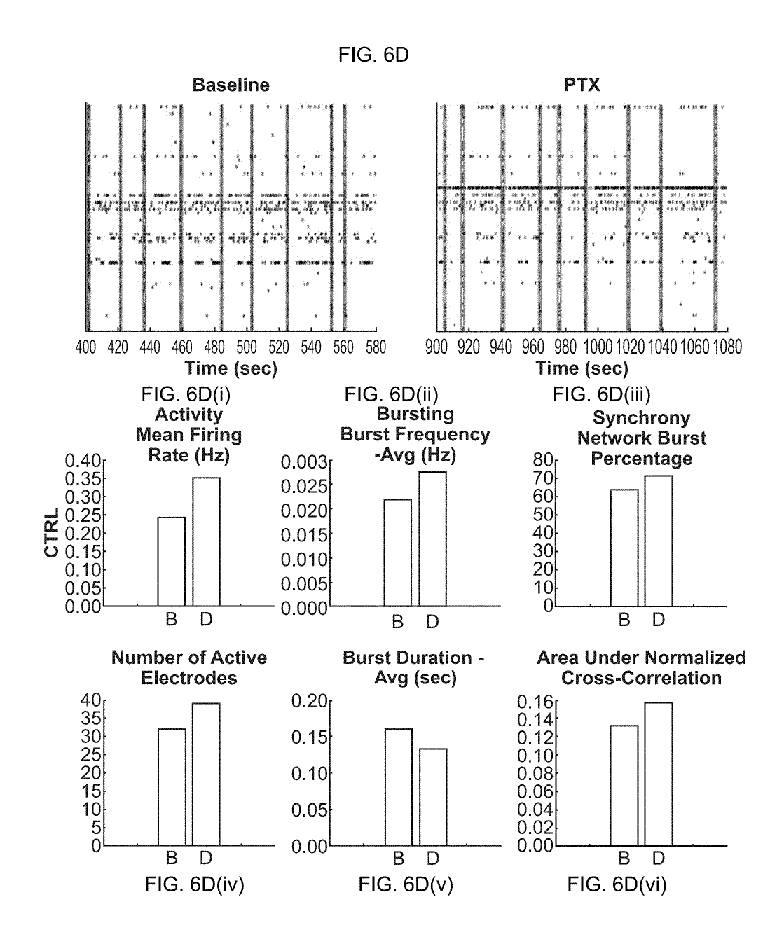

Neural co-cultures were produced as described under Example 1. Neuronal activity was recorded using the Axion BioSystems MEA system set to detect neural spikes applying a bandpass filter from 200 Hz-3 kHz (Neural Spikes mode) and the threshold values for defining active electrodes, bursts and spontaneous synchronized network activity were used as previously described (Example 1). Recordings were performed at week 4 in culture for 10 minutes for each condition including baseline measurements prior to compound application and test measurements following the addition of a single compound. Between baseline and test condition recordings, the plates were store in an incubator (37° C., 5% CO₂) to readjust pH of the media. Each compound was applied to a different well in order to prevent secondary effects of previous treatments. First, the effect of the common compound solvent DMSO was tested by adding 0.1% to the neural co-culture medium. As depicted in FIG. 6A i and ii, DMSO alone increased the frequency of total spikes (mean firing rate) and the frequency of total bursts by 0.2 Hz and 0.018 Hz, respectively. Moreover, a minor increase in synchrony could be detected increasing the percentage of bursts within synchronized network activity by approximately 5%. This suggested that DMSO increases neuronal activity to a minor extent, which needs to be considered for subsequent experiments.

Second, the effect of CNQX, a competitive inhibitor of AMPA receptors, was tested by adding a final concentration of 20 μm to the neural co-culture medium. As shown in FIG. 6B i and ii, CNQX decreased the frequency of total spikes (mean firing rate) and the frequency of total bursts by 0.4 Hz and 0.005 Hz, respectively. Strikingly, CNQX application completely abolished the occurrence of synchronized network activity (FIG. 6B, iii). These data confirm the expected inhibitory effect on glutamatergic excitatory neurons leading to significant decrease in neuronal activity and complete suppression of spontaneous synchronized network activity by blocking glutamatergic synaptic transmission.

Third, the effects of AP5, a selective inhibitor of NMDA receptors, was tested by adding a final concentration of 50 μm to the neural co-culture medium. As shown in FIG. 6C, AP5 decreased the frequency of total spikes (mean firing rate) and the frequency of total bursts by 0.2 Hz and 0.004 Hz, respectively. Moreover, the average duration of bursts was reduced by about 30% (FIG. 6C, v) indicating an impairment of sustained excitability by compromising the role of NMDA receptors in producing stimulus train-induced bursting. In accordance to this, synchronized network activity was also reduced upon addition of AP5 as measured by network burst percentage (FIG. 6C, iii).

Fourth, the effects of PTX, a non-competitive inhibitor of GABAA receptors, was tested by adding a final concentration of 50 μm to the neural co-culture medium. Since the neural co-culture assessed in this experiment consisted of pure glutamatergic excitatory iN cells and did not include GABAergic inhibitory cells, inhibition of GABA receptors was not expected to show and any significant effect. As shown in FIG. 6D, the observed increase in spike and burst frequencies, as well as the minor increase in synchronized network activity largely corresponded to the changes observed upon application of 0.1% DMSO, which also served as a solvent for PTX (compare FIG. 6A). These data confirm the absence of inhibitory neurons and demonstrates the purity of generating specified neuronal subtypes by the method described herein.

### Example 4

### Effects of Chemical Compounds on Neuronal Network Activity in Neural Co-Cultures Consisting of Primary Glial Cells and a Mixture of Glutamatergic Excitatory iN Cells and GABAergic Inhibitory iN Cells Measured on MEAs

Neural co-cultures were produced as described under Example 2 resulting in excitatory/inhibitory iN cell ratio of approximately 70%/30%. Recordings of neuronal activity, threshold values, and the procedure of measuring baseline and test conditions were performed as described under Example 3. Here, the effect of PTX was tested by adding a final concentration of 50 μm to the neural co-culture medium.

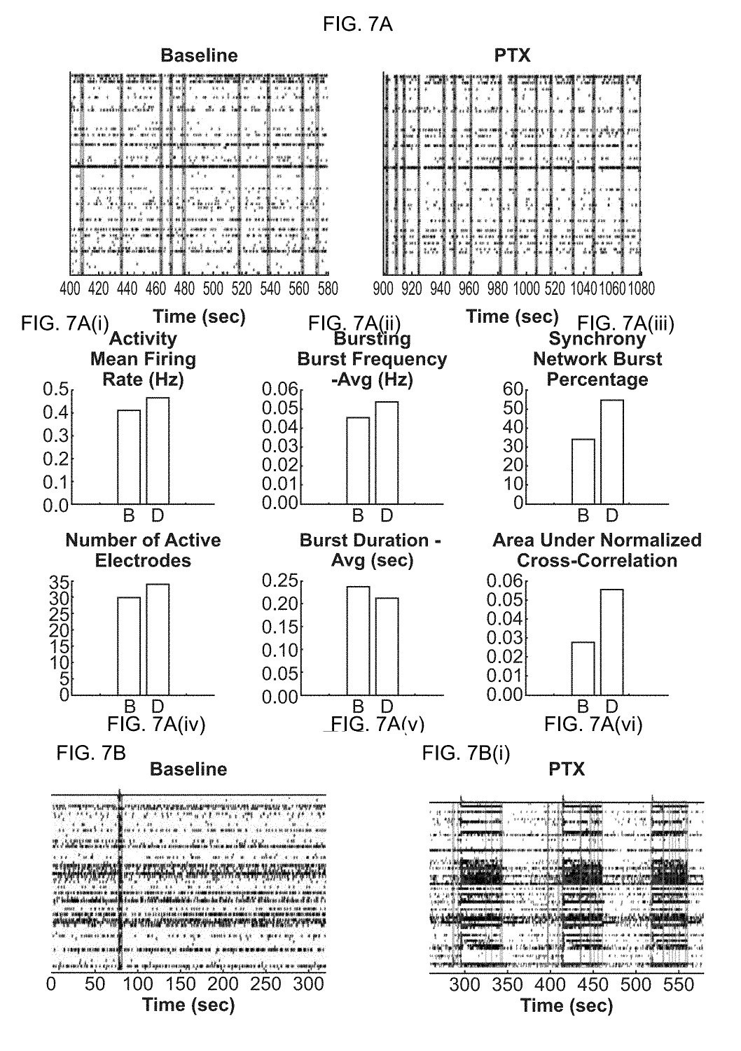

As shown in FIG. 7A i and ii, an increase in spike and burst frequencies was observed, which resembled the observation in PTX-treated pure excitatory neural co-culture likely reflecting the unspecific effect of the solvent DMSO (compare FIG. 6A). However, in contrast to pure excitatory cultures, the synchrony of network bursts was increased by almost 60% (FIG. 7A, iii) upon PTX application suggesting an enhancement of synchronized network activity through blocking the inhibitory component of the culture. These data demonstrate that the inhibitory component functionally contributes to the properties of the network activity.

In a second experiment, the effect of PTX on synchronized neuronal network activity in neural co-cultures with a stronger inhibitory component was assessed. For this purpose, a total of 100,000 excitatory iN cells and 300,000 inhibitory iN cells were plated simultaneously per well on 12-well MEA plates (Axion BioSystems) coated with matrigel. Glial cells were derived from human primary NSCs as described above and seeded directly on the plated iN cells at a density of 100,000 cells per well. Due to the different apoptosis rates of the two iN cells types mentioned earlier, the final ratio of excitatory/inhibitory neurons after 4 weeks in culture was approximately 50%/50%.

The effect of PTX was tested by adding a final concentration of 50 μm to the neural co-culture medium. As shown in FIG. 7B i, baseline synchronized network activity was strongly reduced compared to baseline measurements of mixed co-cultures with higher excitatory/inhibitory ratios (compare FIG. 5). Upon PTX treatment, extended synchronized bursts with shortening inter-burst intervals were detected resembling ictal foci of epileptic seizure activity patterns (FIG. 7B, ii). These data demonstrate that the provided neural screening system can be applied to study seizure-like phenotypes as well as effect of convulsant and anticonvulsant agents.

### Example 5

### Synchronized Network Activity in Neural Co-Cultures Containing either Primary Glial Cells Derived from Mice or Human Glial Cells Differentiated from Early Glial Progenitors

Excitatory iN cells were produced as described above. Human glial cells were produced by differentiation of early glial progenitors using neural medium (neural medium: 500 ml DMEM/F12, 5 ml N2 supplement, 5 ml MEM non-essential amino acids, 1 ml heparin [1 mg/ml]) supplemented with 10 ng/ml EGF and 3% fetal bovine serum). Excitatory iN cells were plated on 12-well MEA plates as described above. Primary mouse glial cells were produced and seeded as stated under Example 1 at a total density of 100,000 cells per well. Progenitor-derived human glial cells were seeded directly on pre-plated iN cells at a comparable density. In addition, iN cells and either mouse or human glial cells were plated accordingly on matrigel-coated cover slips for patch clamp analysis. Recordings of neuronal activity using MEAs were performed as described under Example 3 at 4 weeks after plating. Patch clamp analyses were conducted to measure spontaneous excitatory postsynaptic currents (EPSCs) from single neurons.

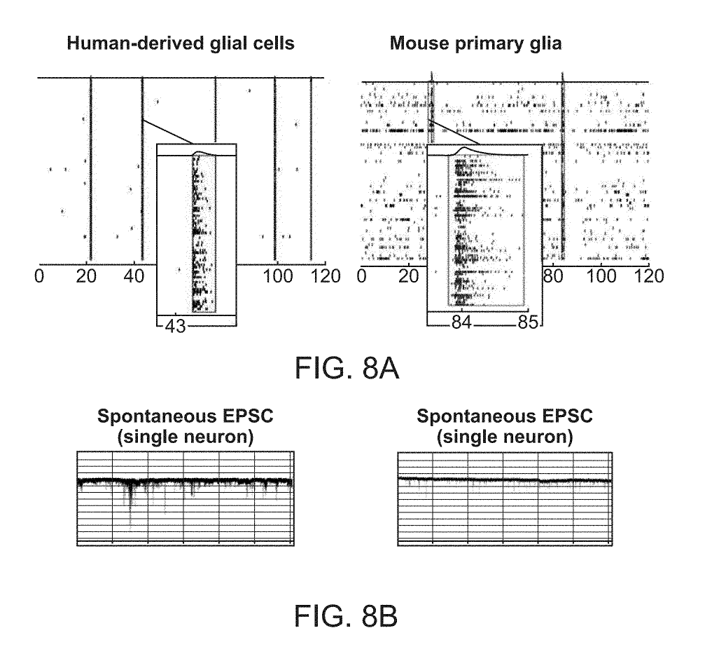

As depicted in FIG. 8A, neural co-cultures comprising human glial cells exhibited synchronized network activity with significant higher frequencies as compared to co-cultures comprising primary mouse glial cells. Moreover, the total number of spontaneous single spikes between network bursts was drastically reduced in pure human neural co-cultures indicating a more mature neuronal network with less uncoordinated firing of immature neurons that are not integrated in the network. Interestingly, the duration of individual network burst were decreased in pure human versus mixed human/murine co-cultures (FIG. 8A, magnification boxes). Patch clamp analyses further showed markedly increased EPSCs both in frequency and amplitude in pure human cultures demonstrating pronounced synaptic function. These data suggest that pure human neural co-cultures using human astroglial cells support neuronal maturation and produce neuronal networks with higher activity and less noise compared to mixed human/murine co-cultures. Therefore, pure human neural co-cultures comprising human glial cells and fast maturing, directly induced neurons are suitable for assessment of complex neuronal activity on non-invasive, integral monitoring platforms to constitute a new neural screening system as provided by this invention.

### Example 6

### Effects of Well-Known Neurotoxic Chemicals on Neuronal Network Activity in Neural Co-Cultures Consisting of Primary Human Glial Cells and a Mixture of Glutamatergic Excitatory iN Cells and GABAergic Inhibitory iN Cells Measured on MEAs

Induced excitatory neurons were seeded at day 4 and induced inhibitory neurons were seeded at day 6 after their induction. A total of 140 000 excitatory iN cells and 60 000 inhibitory iN cells were plated with 70 000 primary human astroglial cells per well on 48-well MEA plates (Axion BioSystems) coated with PEI/laminin. All cells were seeded simultaneously in neuronal seeding media (Neurobasal-A medium, B27 supplement, Glutamax [1 mM], NT3 [10 ng/ml], mouse laminin [200 ng/ml], doxycycline [2 μg/ml], and 1% FBS) and cultured at 37° C. and 5% CO₂. At 2 days after seeding, media was switched to neuronal maintenance media (Neurobasal-A medium, B27 supplement, Glutamax [1 mM], NT3 [10 ng/ml], doxycycline [2 μg/ml], mouse laminin [200 ng/ml], AraC [2 μm], 1% FBS) and half media changes were performed every 3 days.

At 40 days after plating, baseline recording was performed for 30 minutes. Subsequently, 12 different compounds and solvent controls were applied in 7 different concentrations with 6 technical replicates per condition. This included 3 negative controls (amoxicillin, salicylic acid, and glyphosate), 1 positive control for neuronal cytotoxicity (tributyltin), and 8 well-characterized neurotoxic chemicals (picrotoxin, bicuculline, lindane, dieldrin, deltamethrin, permethrin, esfenvalerate, and cypermethrin). After compound dosing, neural co-cultures were left for equilibrated for 30 minutes before recording neuronal activity for another 30 minutes. In parallel, primary cortical rat cultures were prepared as described in Wallace et al. (Ref: Wallace, Strickland, Valdivia, Mundy, Shafer, NeuroToxicology 2015) and treated accordingly.

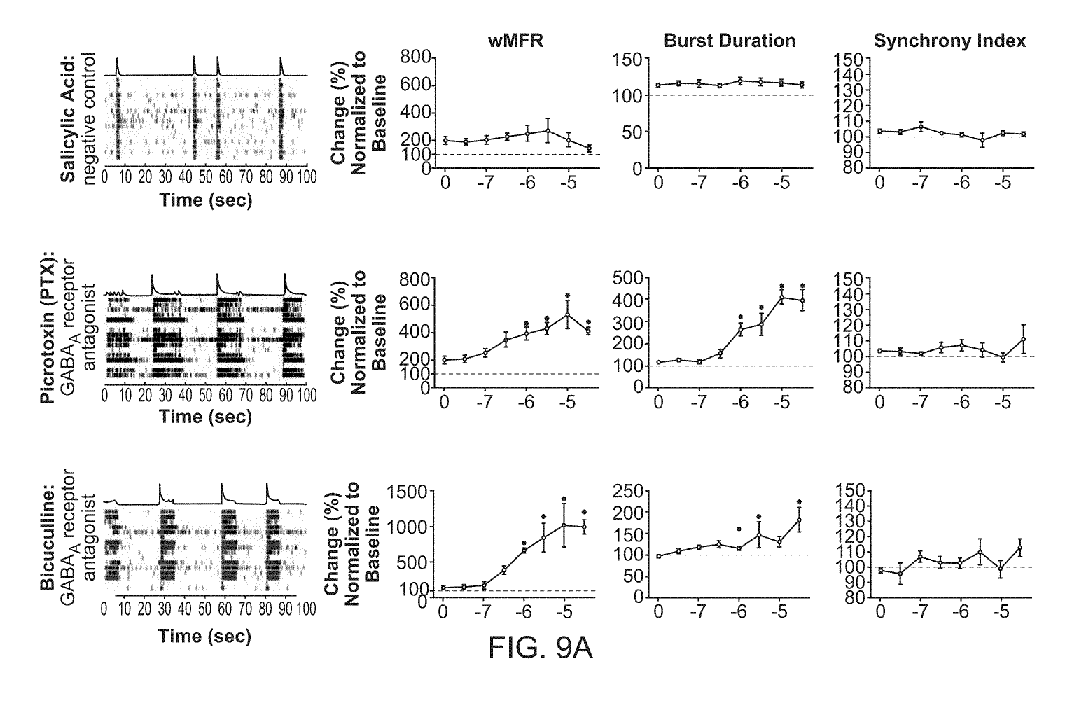

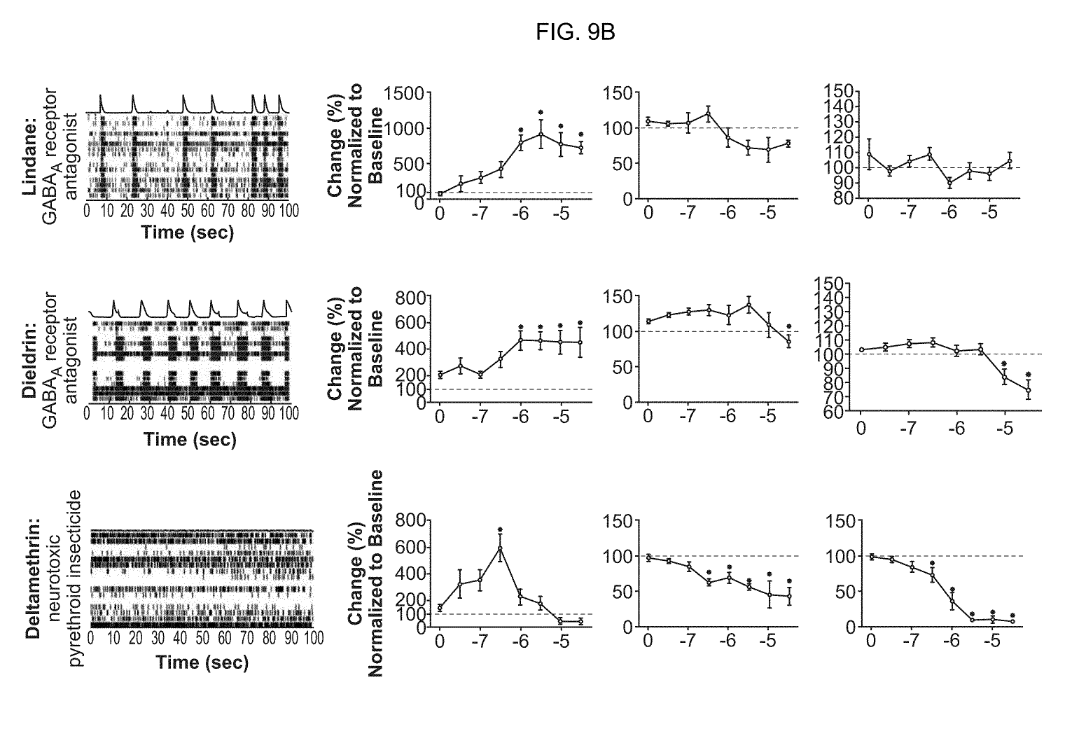

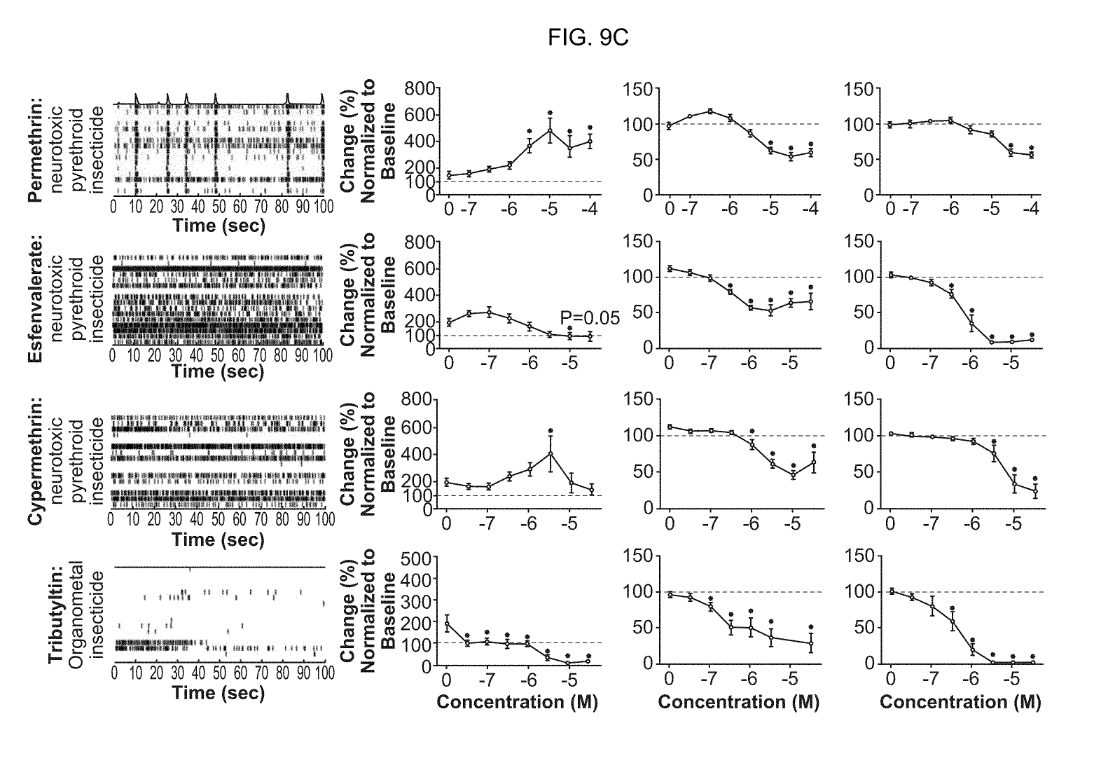

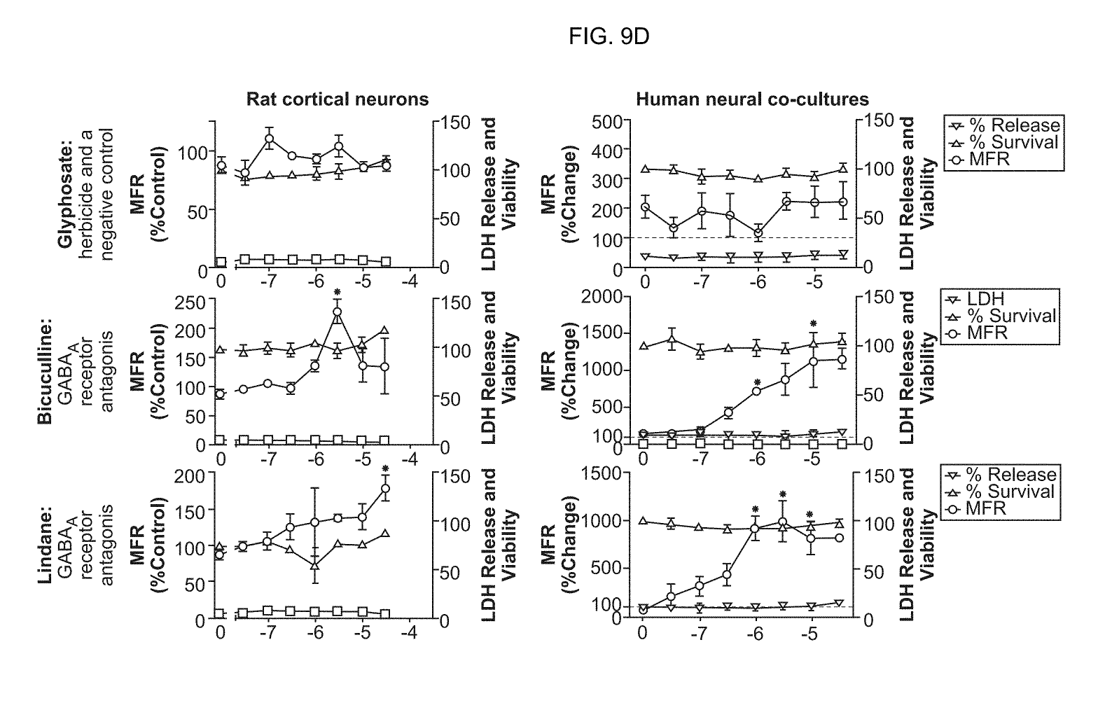

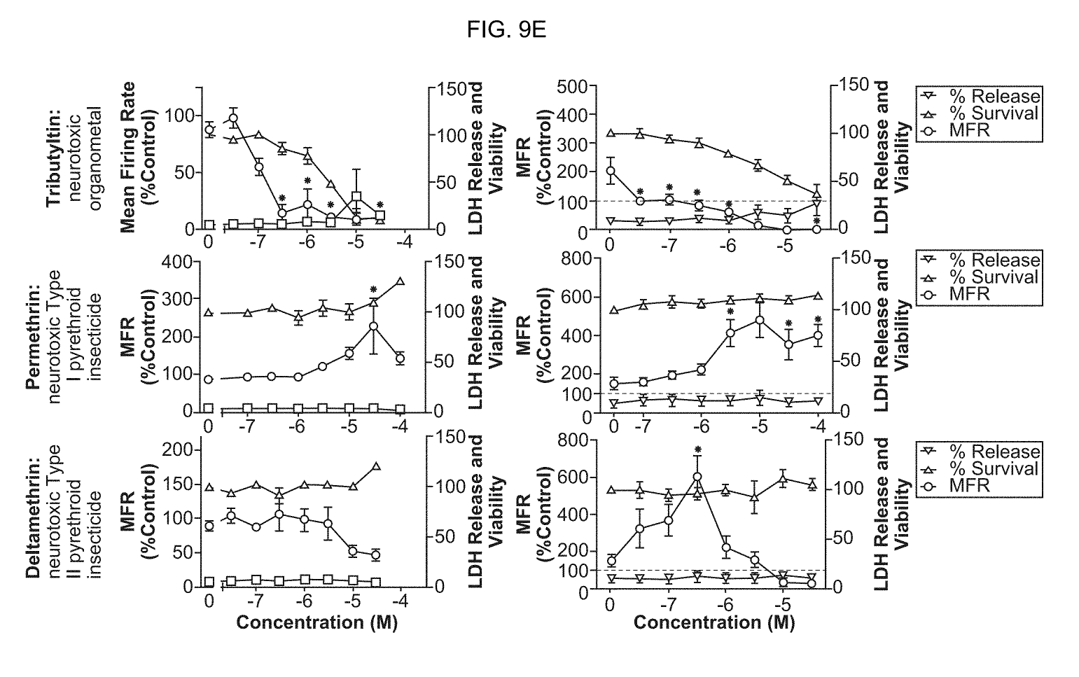

Neuronal activity was recorded using the Axion BioSystems MEA system set to detect neural spikes applying a bandpass filter from 300 Hz-5 kHz (Neural Spikes mode) and a detection threshold of 8-fold standard deviation. Here, only wells that show 8 or more active electrodes (at least 5 spikes detected per minute) per well (out of 12) were regarded for evaluation. Data analysis were carried out using AxIS and Neurometric tool from Axion Biosystems. As shown in FIG. 9Bi, control compounds did not significantly alter any parameters related to single spiking, bursting, coordinated network activity in human neural co-cultures. In contrast, compounds of the GABAA-antagonist class increase neuronal activity with typical changes in network burst behavior leading to extended highly synchronous discharges (FIG. 9Aii-v). Neurotoxic compounds of the second class, type-I and type-II pyrethroids, mostly increased overall spiking activity but mostly disrupted network coordination resulting significant decrease in synchrony parameters in a dose-dependent manner (FIG. 9Avi-ix). Moreover, a direct comparison of changes of neuronal activity upon compound application between rodent primary and human iN co-cultures revealed high concordance in dose-dependent phenotypes (FIG. 9B). Of note, except for tributyltin, none of the tested compound significantly affected cell viability as measured by LDH release and cell titer blue assays (FIG. 9B). These results demonstrate the usability of the described neural co-cultures for identifying and describing neurotoxic effects of different compound classes. These data demonstrate that mixed excitatory/inhibitory neural co-cultures are capable of forming spontaneous synchronized network activity.

### Example 7

### Effects of Antiepileptic Drugs (AEDs) on Chemically Induced Seizure-Like Activity Patterns of Network Activity in Neural Co-Cultures Consisting of Primary Human Glial Cells and a Mixture of Glutamatergic Excitatory iN Cells and GABAergic Inhibitory iN Cells Measured on MEAs

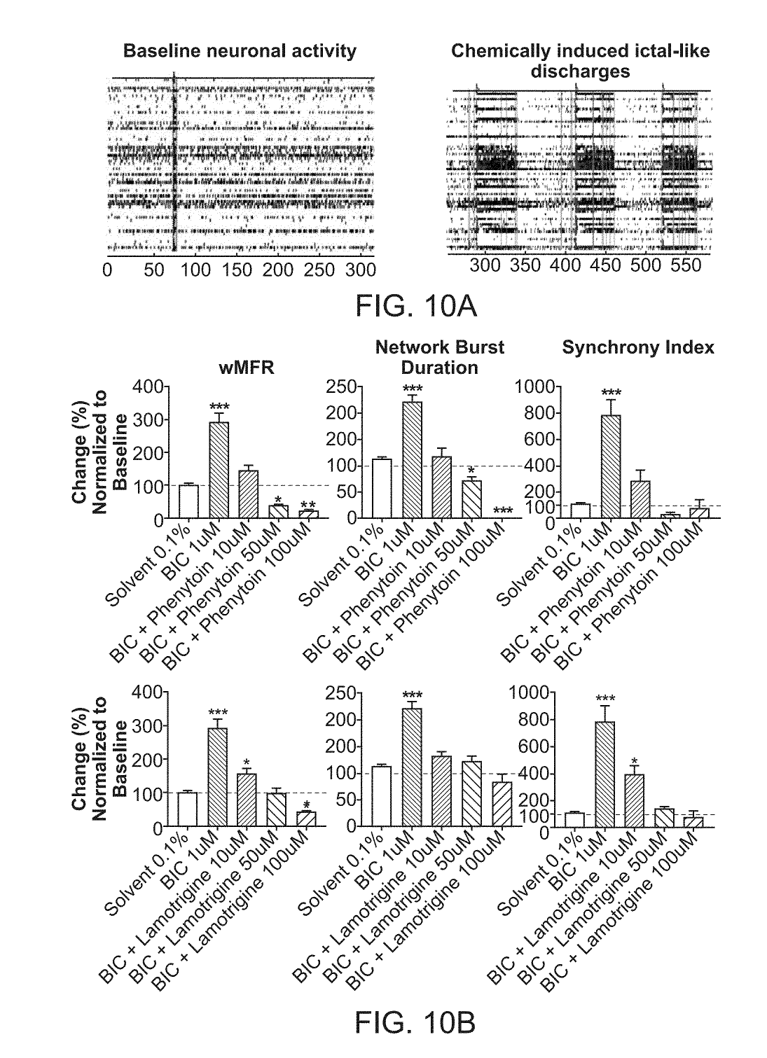

Generation of human neural co-cultures and MEA recording settings and thresholds were as described under Example 6. At 24 days after plating, the GABAA-antagonist and well-characterized proconvulsive compounds bicuculline was applied at 1 μm concentration to induce ictal-like discharges to model synapse-dependent seizure activity, which is generally characterized by extended, highly synchronous network bursts (FIG. 10A). Importantly, co-application of the FDA-approved AEDs phenytoin or lamotrigine reduced critical parameters of general neuronal activity such as mean firing rate, network burst duration, and synchrony to baseline level (solvent control), or further reduced the same parameters in a dose-dependent manner (FIG. 10B). Co-application of AEDs was performed in 3 different doses and each condition was tested in 6 technical replicates. These results indicate the usability of the described human neural co-cultures system combined with MEA readouts to serve as a screening platform for proconvulsive countermeasures and antiepileptic drugs.

### REFERENCES

Culbreth M E, Harrill J A, Freudenrich T M, Mundy W R, Shafer T J: Comparison of chemical-induced changes in proliferation and apoptosis in human and mouse neuroprogenitor cells. Neurotoxicology 2012, 33(6):1499-1510.
Saeedi Saravi S S, Dehpour A R: Potential role of organochlorine pesticides in the pathogenesis of neurodevelopmental, neurodegenerative, and neurobehavioral disorders: A review. Life Sci 2016, 145:255-264.
Boyden E S, Zhang F, Bamberg E, Nagel G, Deisseroth K: Millisecond-timescale, genetically targeted optical control of neural activity. Nat Neurosci 2005, 8(9):1263-1268.
Han X, Chow B Y, Zhou H, Klapoetke N C, Chuong A, Rajimehr R, Yang A, Baratta M V, Winkle J, Desimone R et al: A high-light sensitivity optical neural silencer: development and application to optogenetic control of non-human primate cortex. Front Syst Neurosci 2011, 5:18.
Michaelson J J, Shi Y, Gujral M, Zheng H, Malhotra D, Jin X, Jian M, Liu G, Greer D, Bhandari A et al: Whole-genome sequencing in autism identifies hot spots for de novo germline mutation. Cell 2012, 151(7):1431-1442.
Ripke S, O'Dushlaine C, Chambert K, Moran J L, Kahler A K, Akterin S, Bergen S E, Collins A L, Crowley J J, Fromer M et al: Genome-wide association analysis identifies 13 new risk loci for schizophrenia. Nat Genet 2013, 45(10):1150-1159.
Xu B, Ionita-Laza I, Roos J L, Boone B, Woodrick S, Sun Y, Levy S, Gogos J A, Karayiorgou M: De novo gene mutations highlight patterns of genetic and neural complexity in schizophrenia. Nat Genet 2012, 44(12):1365-1369.
Walker F O: Huntington's disease. Lancet 2007, 369(9557):218-228.
Martin I, Dawson V L, Dawson T M: Recent advances in the genetics of Parkinson's disease. Annu Rev Genomics Hum Genet 2011, 12:301-325.
Schizophrenia Psychiatric Genome-Wide Association Study C: Genome-wide association study identifies five new schizophrenia loci. Nat Genet 2011, 43(10):969-976.
Dunham I, Shimizu N, Roe B A, Chissoe S, Hunt A R, Collins J E, Bruskiewich R, Beare D M, Clamp M, Smink L J et al: The DNA sequence of human chromosome 22. Nature 1999, 402(6761):489-495.
Deloukas P, Schuler G D, Gyapay G, Beasley E M, Soderlund C, Rodriguez-Tome P, Hui L, Matise T C, McKusick K B, Beckmann J S et al: A physical map of 30,000 human genes. Science 1998, 282(5389):744-746.
Rodriguez-Frade J M, Vila-Coro A J, de Ana A M, Albar J P, Martinez A C, Mellado M: The chemokine monocyte chemoattractant protein-1 induces functional responses through dimerization of its receptor CCR2. Proc Natl Acad Sci USA 1999, 96(7):3628-3633.
Mochly-Rosen D: Localization of protein kinases by anchoring proteins: a theme in signal transduction. Science 1995, 268(5208):247-251.
Jones K, Hibbert F, Keenan M: Glowing jellyfish, luminescence and a molecule called coelenterazine. Trends Biotechnol 1999, 17(12):477-481.

1. A human neural cell co-culture that provides synchronous network bursts, the co-culture comprising:
in vitro differentiated functional human neuronal cells; and
human glial cells.

2. The neural cell co-culture of claim 1, wherein the in vitro differentiated functional human neuronal cells are derived by the method comprising:
contacting a population of non-neuronal human cells with neuron reprogramming factors (NR), or agents to activate NR factors, wherein the NR factors are selected from the group consisting of: Neurogenin, Ascl, NeuroD, Brn2, Brn3a, Emx, Cux2, Tbr1, Satb2, Dlx1/2/5, Nkx2.1, Nkx2.2, Lhx2/3/6/8, Sox2, Foxg1, Ctip2, Hb9, Isl1/2, Klf7, Gata2, Foxa2, Lmx1b, Ptx, FEV, Lmx1, Foxa2, Nurr1, Pitx3, and En for a period of time sufficient to reprogram said non-neural cells,
wherein a population of functional human neuronal cells is produced.

3. The neural cell co-culture of claim 1, wherein the non-neuronal cells are pluripotent cells.

4. The neural cell co-culture of claim 1, wherein the non-neuronal cells are somatic cells.

5. The neural cell co-culture of claim 1, wherein the non-neuronal cells are somatic stem cells.

6. The neural cell co-culture of claim 1, wherein the neuronal cells are iN cells.

7. The neural cell co-culture of claim 1, wherein the neuronal cells comprise one or more of GABAergic inhibitory neurons, glutamatergic excitatory neurons, dopaminergic excitatory neurons, and serotonergic neurons.

8. The neural cell co-culture of claim 1, wherein the human glial cells are derived by the method comprising:
isolating glial cells from primary brain tissue.

9. The neural cell co-culture of claim 1, wherein the human glial cells are derived by the method comprising:
contacting a population of non-glial cells with one or more of whole serum, single serum components, insulin, BMP-inhibitor, TGF-□□inhibitor, EGF, CNTF, BMP2/4, NFIA, NFIB, SOX9, and HES for a period of time sufficient to reprogram or step-wise differentiate non-glial cells to astroglial cells.

10. The neural cell co-culture of claim 9, wherein the non-glial cells are pluripotent cells.

11. The neural cell co-culture of claim 9, wherein the non-glial cells are somatic cells.

12. The neural cell co-culture of claim 9, wherein the non-glial cells are neural stem cells.

13. The neural cell co-culture of claim 1, wherein the neuronal and/or glial cells are derived from healthy individuals.

14. The neural cell co-culture of claim 1, wherein the neuronal and/or glial cells are derived from individuals diagnosed with a disease of interest.

15. The neural cell co-culture of claim 1, wherein the neuronal and/or glial cells are genetically modified to introduce or remove genetic causes of a disease phenotype.

16. The neural cell co-culture of claim 1, wherein in a panel of co-cultures the neuronal and/or glial cells are derived from multiple individuals.

17. A system for biologically relevant screening of neuronal activity, comprising:
a human neural cell co-culture according to claim 1; and
a monitoring device.

18. The system of claim 17, wherein the monitoring device comprises a multielectrode array.

19. The system of claim 17, wherein the monitoring device provides optical signal detection with one or more of calcium indicators and voltage-sensitive dyes.

20. A method for biologically relevant screening of altered neuronal function, the method comprising:
contacting a system according to claim 17 with an agent and determining a change in at least one neuronal parameter.

21. A method for biologically relevant screening of altered neuronal function, the method comprising:
stimulating or perturbing components of a system according to claim 17 with electrical or optogenetic means and determining a change in at least one neuronal parameter.

22. The method of claim 20, wherein neuronal parameters comprise one or more of: neuronal viability; total number of spikes (per recording period); mean firing rate (of spikes); inter-spike interval (distance between sequential spikes); total number of bursts (per recording period); burst frequency; number of spikes per burst; burst duration (in milliseconds); inter-burst interval (distance between sequential bursts); burst percentage (the portion of spikes occurring within a burst); total number of network bursts (spontaneous synchronized network activity); network burst frequency; number of spikes per network burst; network burst duration; inter-network-burst interval; inter-spike interval within network bursts; network burst percentage (the portion of bursts occurring within a network burst); and cross-correlation of detected spikes between all electrodes per well.

23. The method of claim 20, wherein the agent is a candidate therapeutic agent.

24. The method of claim 20, wherein the agent is a genetic agent.

25. The method of claim 20, wherein the agent is a known neurotoxin and a candidate antagonist to the neurotoxin.
